SEQUENCE LISTING (19 sequences)

SEQ ID NO: 1 - protein, 477 aa, synthetic construct
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFEYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 2 - DNA, 1431 bp, synthetic construct
  source 1..1431: mol_type = other DNA
atggccaccccatccgcattctccggcagcgtcctcacccccggtgacgacggcttcgaggcggcccaggtcacctggaacgcctgctactcctcccgcccccgggaggtgatggtctgccacgacgccgcgtccgtcgccgaggcggtgcgcagcgtgcgggagcgcggactgcccttccgggtgcgctccggcggccactcgatgtgcgggctgagcaacctggacgacggcgtcatcatcgacctcggcggcctgggcggcgtcgaactcaccccggaccggcagacggtgcggatcggcggcggcgcccgcctcgccgacgtctacaacaccctctgggaccaccggctgaccgtcccggccggcacctgcccgcggatcggggtcggcggccacgtgctgggcggcggcatgggcgtgctgtcccggtcgcgtggcgcgctggtggaccatctgacggcactggagatggtggacgccgagggccggctcctgcgcgtcagcgaggacgagaaccccgacctgttctgggcctgccggggcggcggtggcggcaacttcggcatcgtgaccgcgtacgagctgcggcccacgccgatcgacgacgtcaccatcttcacggtcagctggacctggtcgcagctccccgacgcggtgcgggcctggcagcggtggctgggctcggcggagagccgtatcaacagcttcctctcactcttcccccaacagcaggacatggtcgtcgcgttcggcgtcttcgacggtccggccgccgacttccggccgctgctggccccgctgaccgccgaggtcgccccggaggccgaggtcgtggaggaggtcccgttcatccaggccgtggacaccgtcgaggcgttgcagggcgaggccgccgccgccgagcaggtgcgggcgcagggcagttcggcgatcatcgcgaacccgctgaacgacgaggcgctggccacgctccaggagttcctcaccgacccgcccagccaccgagcggaggtggccgtctacgggatgggcggcgtcatcggcgaacgggagcgcggcgacacggcgttcgtgcaccgcaccggcctgatggccttcgagtaccgcaccgactgggacagccccgaggacgaccggctcaacctggactgggtgacccggctgcggcacgcgatggccgaacacaccaccggcgccgcctacgtcaacaccatcgacctggccctggagaactggctgtgggcctactacgaggagaacctgccgcgcctgatggcggtcaagcggcggtacgacccggagaacgtcttccaccacccgcacagcatccccggctcgctcaccgccgaggcggcccgggcgcacggcgttcccgaggccaccctcaagcggctgcacgacgacggcctgctcgacggaccgctggac

SEQ ID NO: 3 - protein, 477 aa, synthetic construct
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFEYRTDWDSPEDDRLNLDWVTRLRHAMAEHTTGAAYVNYIDLALENWLWAYYEENLPRLMAVKRRYDPENVFHHPHSIPGSLTAEAARAHGVPEATLKRLHDDGLLDGPLD

SEQ ID NO: 4 - protein, 477 aa, synthetic construct
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGFCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFEYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 5 - protein, 477 aa, synthetic construct
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFNYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 6 - protein, 506 aa, synthetic construct
VTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 7 - DNA, 678 bp, synthetic construct
  source 1..678: mol_type = other DNA
gccgcgctcggctgcttcgagggcgacgcggccgaactggagccgctcatcgcaccgttggtgcacgccgtcgagccgaccgagaaggtcgccgagaccatgccctggatcgaggcgctctccttcgtggagaccatgcagggcgaggccatgaaggccacctcggtacgggcgaagggcaacctctcgttcgtgacggaaccgctgggcgacagggccgtcgaggagatcaagaaggccctcgcccaagcccccagccaccgcgccgaggtggtcctctacgggctcggcggcgccgtcgcggccaagggccgccgggagaccgcgttcgtccaccgggacgcccccgtcgcgctcaactaccacaccgactgggacgacgaggcggaggacgacctcaacttcgcctggattcagaacctgcgggcaagcgtcgccgcgcacaccgaaggcaggggcagctacgtcaacaccatcgacctgaccgtcgagcactggctctgggactactacgaggagaacctgccccggctgatggcggtcaagaagcggtacgacccggaggacgtcttccgccacccgcagagcatccccgtctcgctcaccgaggcggaggcggccgaactggggattccgccgcacatcgccgaggaactccgggccgccaggcagctgcgg

SEQ ID NO: 8 - protein, 506 aa, synthetic construct
VTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGTCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 9 - protein, 506 aa, synthetic construct
VTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNYIDLTVEHWLWDYYEENLPRLMAVKKRYDPEDVFRHPQSIPVSLTEAEAAELGIPPHIAEELRAARQLR

SEQ ID NO: 10 - DNA, 19 bp, synthetic construct
  source 1..19: mol_type = other DNA
ggccggcacctgcccggag

SEQ ID NO: 11 - DNA, 25 bp, synthetic construct
  source 1..25: mol_type = other DNA
cgtcgcgctcgaataccacaccgac

SEQ ID NO: 12 - DNA, 25 bp, synthetic construct
  source 1..25: mol_type = other DNA
cgtcgcgctcctgtaccacaccgac

SEQ ID NO: 13 - DNA, 33 bp, synthetic construct
  source 1..33: mol_type = other DNA
gcagctacgtcaactatatcgacctgaccgtcg

SEQ ID NO: 14 - DNA, 20 bp, synthetic construct
  source 1..20: mol_type = other DNA
ccggccggcttttgcccgcg

SEQ ID NO: 15 - DNA, 20 bp, synthetic construct
  source 1..20: mol_type = other DNA
cgcgggcaaaagccggccgg

SEQ ID NO: 16 - DNA, 29 bp, synthetic construct
  source 1..29: mol_type = other DNA
ctgatggccttcaactaccgcaccgactg

SEQ ID NO: 17 - DNA, 29 bp, synthetic construct
  source 1..29: mol_type = other DNA
cagtcggtgcggtagttgaaggccatcag

SEQ ID NO: 18 - DNA, 29 bp, synthetic construct
  source 1..29: mol_type = other DNA
ccgcctacgtcaactatatcgacctggcc

SEQ ID NO: 19 - DNA, 29 bp, synthetic construct
  source 1..29: mol_type = other DNA
ggccaggtcgatatagttgacgtaggcgg

(19) United States
(12) Patent Application Publication
(10) Pub. No.: US 2025/0027127 A1
(43) Pub. Date: Jan. 23, 2025

(54) CANNABINOID PRODUCTION IN BACTERIA

(71) Applicant: The Regents of the University of California, Oakland, CA (US)

(72) Inventors: Trevor N. Purdy, San Diego, CA (US); Bradley S. Moore, La Jolla, CA (US)

(21) Appl. No.: 18/692,695

(22) PCT Filed: Sep. 15, 2022
(86) PCT No.: PCT/US2022/043696

§ 371 Date: Mar. 15, 2024

Related U.S. Application Data
(60) Provisional application No. 63/244,370, filed on Sep. 15, 2021.

(51) Int. Cl. C12P 17/10; C12N 9/02; C12P 7/42

(52) U.S. Cl. CPC C12P 17/10; C12N 9/0004; C12P 7/42; C12Y 121/03003

(57) ABSTRACT

The disclosure relates to a method of making at least one cannabinoid comprising contacting at least one berberine bridge enzyme like (BBE-like) oxidase with at least one substrate, wherein the substrate comprises a cannabinoid precursor. The cannabinoid precursor can include cannabigerolic acid dihydrotetrachlorizine, prechlorizidine, cannabigerorcinic acid, grifolic acid, or a combination thereof.

### CROSS-REFERENCE TO RELATED APPLICATION

This application is a U.S. National Stage Filing under 35 U.S.C. 371 from International Application No. PCT/US2022/043696, filed on Sep. 15, 2022, and published as WO 2023/043946 A1 on Mar. 23, 2023, which claims the priority of U.S. provisional application Ser. No. 63/244,370, filed Sep. 15, 2021, the disclosure of each of which is incorporated herein by reference in its entirety.

### FEDERAL FUNDING

This invention was made with government support under grant no. R01-A147818 A1047818 awarded by the National Institutes of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Aug. 7, 2024, is name 1133111US1.xml and is 23,232 bytes in size.

### BACKGROUND

Total synthesis has been routinely employed over the past two decades to supply pharmaceutical drugs, fine chemicals, and agrochemicals on an industrial scale.¹ However, most of these industrial processes require toxic reagents, harsh reaction conditions, and routinely struggle to match the selectivity and atom efficiency already designed by nature.

Carbon(sp³)-hydrogen (C—H) bond functionalization is one of the most valuable reactions to install new functionality into the framework of complex natural product scaffolds.¹⁹⁻²¹ However, synthetic approaches have faced challenges in overcoming the high bond dissociation energy of C—H bonds (−100 kcal·mol⁻¹), poor regioselective and stereoselective control, and potential over-oxidation. Benzylic C—H functionalization reactions are slightly more accessible due to their lower bond dissociation energy (−90 kcal·mol⁻¹) and have attracted both synthetic and biocatalytic interest.^{22,27} However, the vast majority of enzymes able to perform such reactions are derived from plant or fungal sources, limiting scalability due to reduced protein expression levels and overall stability.^{35,38}

### BRIEF SUMMARY OF THE INVENTION

Described herein are compositions and methods for making cannabinoids that are useful as therapeutic agents. The compositions include berberine bridge enzyme (BBE)-like oxidases and mutants thereof that catalyze reactions including benzylic hydroxylation and cycloaddition reactions to produce cannabinoids.

In another aspect, provided herein are methods of synthesizing cannabichromenic acid (CBCA) in a bacterium by contacting it or its clarified cell free mixture comprising a cell lysate with cannabigerolic acid (CBGA), wherein the bacterium expresses the BBE-like oxidases Tcz9 or Clz9.

### BRIEF DESCRIPTION OF THE DRAWINGS

FIGS. 1A-B shows active site residues that may be involved in the deprotonation event that differentiates the catalytic function of Tcz9 from Clz9. FIG. 1A shows a model overlay of Clz9 and Tcz9 with THCA synthase (3VTE) with a bicovalently attached FAD cofactor. FIG. 1B shows a graph of HPLC analysis of the in vitro conversion with compound 1 (left) or compound 3 (right) of Scheme 2, comparing the activity of two Clz9 and Tcz9 mutants with their respective wild-type enzymes after 12 hours. UV absorption monitored at 450 nm (left) and 350 nm (right).

FIGS. 2A-D show biocatalytic properties of Tcz9. FIG. 2A shows a graph of an initial HPLC analysis of benzylic hydroxylation reaction in vitro with 2 mol % Clz9 or Tcz9 and compound 8 or compound 9 of Scheme 2 after 12 hours at 37° C. Combined extracted ion chromatogram (EiC) for m/z=216.1, 232.1 [M-H]⁻ (left); m/z=284.0, 300.0 [M-H]⁻ (right). FIG. 2B shows a temperature profile for an in vitro reaction with Tcz9 crude cell lysate and compound 9 of Scheme 2 at pH=7.5 after 8 hours. FIG. 2C is a time course experiment showing optimal product formation after 8-12 hours at 55° C. before significant degradation of the product. FIG. 2D shows the effects of 10-40% DMSO on Tcz9 with compound 9 at 55° C. after 8 hours. Peak area is calculated by integrating the EiC for compound 9 and compound 11 in each sample.

FIG. 3 shows HPLC analysis of the in vitro reactions of cannabigerolic acid (CBGA, compound 12a) with Clz9 or Tcz9 variants after incubation for 12 hours at pH 7.5 and 37° C. No production of tetrahydrocannabinolic acid (THCA, compound 13) or cannabidiolic acid (CBDA, compound 14) was detected at this pH. The major product observed was cannabichromenic acid (CBCA, compound 15a). Compounds 16-18a have yet to be structurally characterized. UV absorption monitored at 300 nm.

FIG. 4 shows HPLC analysis of the in vitro reactions of compound 12b (left) or compound 12c (right) with Clz9 and Tcz9 variants after incubation for 12 hours at pH 7.5 and 37° C. 16-18b-c have yet to be structurally characterized. UV absorption monitored at 300 nm.

FIG. 5 shows HPLC analysis of the in vitro reactions of compound 19 with Clz9 and Tcz9 variants after incubation for 12 hours at pH 7.5 and 37° C. UV absorption monitored at 300 nm.

### DETAILED DESCRIPTION

Flavin adenine dinucleotide (FAD)-dependent oxidoreductases are a family of enzymes that catalyze diverse chemical transformations with high fidelity and biocatalytic utility. The berberine bridge enzyme (BBE)-like subfamily of flavoproteins are noteworthy for their ability to perform a variety of distinctive tailoring reactions in plants, fungi, and bacteria. Notably, BBE-like enzymes are involved in carbohydrate, steroid, meroterpenoid, polyketide, nicotine, cannabinoid, and berberine alkaloid biosynthesis.¹¹⁻¹⁶ These reactions are not only limited to dehydrogenation reactions, but also include challenging carbon-carbon bond forming reactions, such as the namesake berberine bridge forming reaction in berberine-type alkaloids (shown in Scheme 1). BBE-like oxidases share the distinct attribute of bicovalent attachment to flavin adenine dinucleotide (FAD) at the 6- and 8α-positions of the isoalloxazine ring by conserved cysteine and histidine residues, respectively. This feature increases the redox potential of the FAD cofactor, enabling BBE-like oxidases to perform reactions not accessible by other FAD dependent enzymes. Furthermore, structure-function relationship studies have demonstrated that the bicovalent attachment of FAD is involved in enzymatic activity.^{17,18}

Scheme 1. Representative examples of characterized FAD-dependent BBE-like enzymes catalyzing C—C bond forming reactions (1-3) and dehydrogenation reactions (4-6) in natural product biosynthesis.

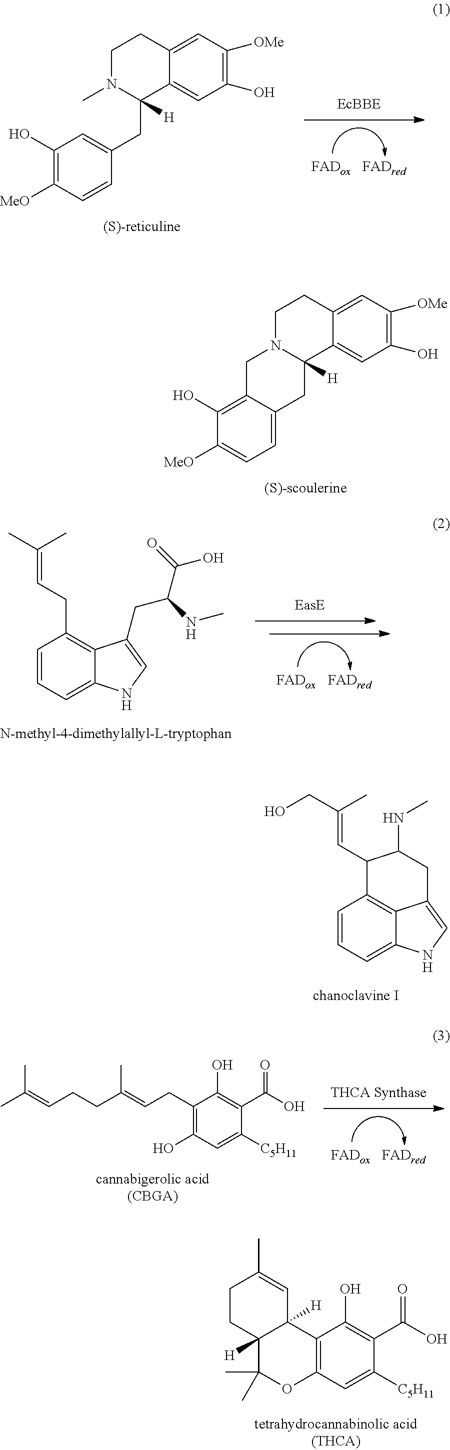

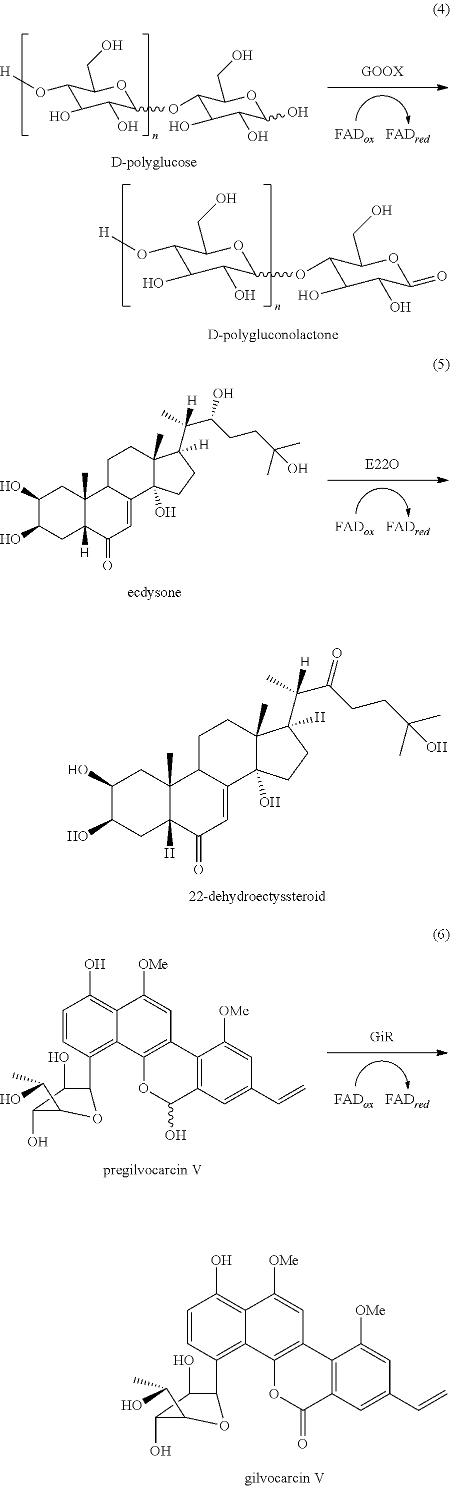

The selective functionalization of benzylic(sp3)C—H bonds remains a formidable task in natural product total synthesis. However, certain enzymes have evolved the ability to perform these reactions under mild reaction conditions.

### Tcz9 and Clz9

A berberine bridge enzyme-like (BBE-like) oxidase, Tcz9, catalyzes a benzylic functionalization reaction via a highly reactive ortho-quinone methide intermediate. A key active site residue was identified that differentiates Tcz9 from Clz9, a homolog with preferential cyclase activity. As described herein, the substrate scope of Tcz9 and Clz9 was expanded to perform benzylic hydroxylation and cycloaddition reactions, including in vitro and in vivo cannabinoid production. Given their favorable biocatalytic properties, Tcz9 and Clz9 highlight new opportunities for engineering related BBE-like enzymes to perform challenging chemo-, regio-, and stereoselective benzylic functionalization reactions.

Herein, two microbial BBE-like oxidases capable of directly generating o-QMs to perform chemoselective dehydrogenation, hydroxylation, and cyclization reactions with an impressive array of non-native substrates are characterized.

A novel tetrachlorinated natural product, tetrachlorizine (compound 4 of Scheme 2 below), was found to be produced by a taxonomically distinct Actinomycete sp.³⁹ Bioinformatic analysis revealed a biosynthetic gene cluster architecturally similar to a previously reported dichloropyrrole containing natural product, (−)-chlorizidine A (compound 2 of Scheme 2 below). The final biosynthetic step in chlorizidine and tetrachlorizine biosynthesis involves the BBE-like oxidases Clz9 and Tcz9, respectively, which share 48% amino acid sequence identity. These two enzymes facilitate hydride abstraction by the oxidized flavin cofactor and phenolic deprotonation by basic residues in the active site, generating a highly reactive o-QM intermediate. There is evidence indicating that this mechanism may be concerted.⁴⁰ Prechlorizidine (compound 1 of Scheme 2) is converted to (−)-chlorizidine A (compound 2 of Scheme 2) by intramolecular nucleophilic addition of the dichloropyrrole nitrogen, generating a rare dihydropyrrolizine ring enantioselectively, while dihydrotetrachlorizine (compound 3 of Scheme 2) is converted to tetrachlorizine (compound 4 of Scheme 2) by benzylic dehydrogenation.

Experiments exploring this difference in catalytic function began by incubating compound 3 with Clz9, yielding a I:I mixture of compound 4 and a new cyclized derivative (compound 5), indicating that dehydrogenation can still occur, but Clz9 preferentially acts as a cyclase (Scheme 2b). In contrast, incubation of prechlorizidine (compound 1) with Tcz9 generated two triply oxidized, stabilized o-QM isomers (compound 6 and 7) that were unexpectedly stable in aqueous solutions at room temperature. The production of compound 6 and 7 indicates Tcz9 is also capable of both intramolecular cyclization and dehydrogenation reactions with non-native substrates, however, the additional dehydrogenation activity with prechlorizidine distinguishes Tcz9 from Clz9.

Products were also observed to undergo multiple iterations of oxidation by either BBE-like enzyme. The C-15 carbonyl of compound 3 may lower the pKa of the α-protons attached to facilitate deprotonation and subsequent dehydrogenation by Tcz9 and Clz9 following o-QM formation. In addition, an active site residue in Tcz9 may be capable of deprotonating C-14 without the C-15 carbonyl due to the absence of any cyclized product with compound 3 and the production of triply dehydrogenated products compounds 6 and 7. Given the results with two substrates and differences in activity between Clz9 and Tcz9, the structure-function relationships of these two enzymes were further elucidated and their substrate promiscuity assessed to functionalize a broader range of substrates.

Scheme 2. Benzylic functionalization reactions with microbial BBE-like enzymes Clz9 and Tcz9 via an o-QM intermediate. (a) Clz9 catalyzes intramolecular cyclization, while Tcz9 catalyzes a dehydrogenation. (b) Clz9 exhibits selectivity as a cyclase not observed with Tcz9 and compound 3, while Tcz9 has preferential dehydrogenation activity not observed with Clz9.

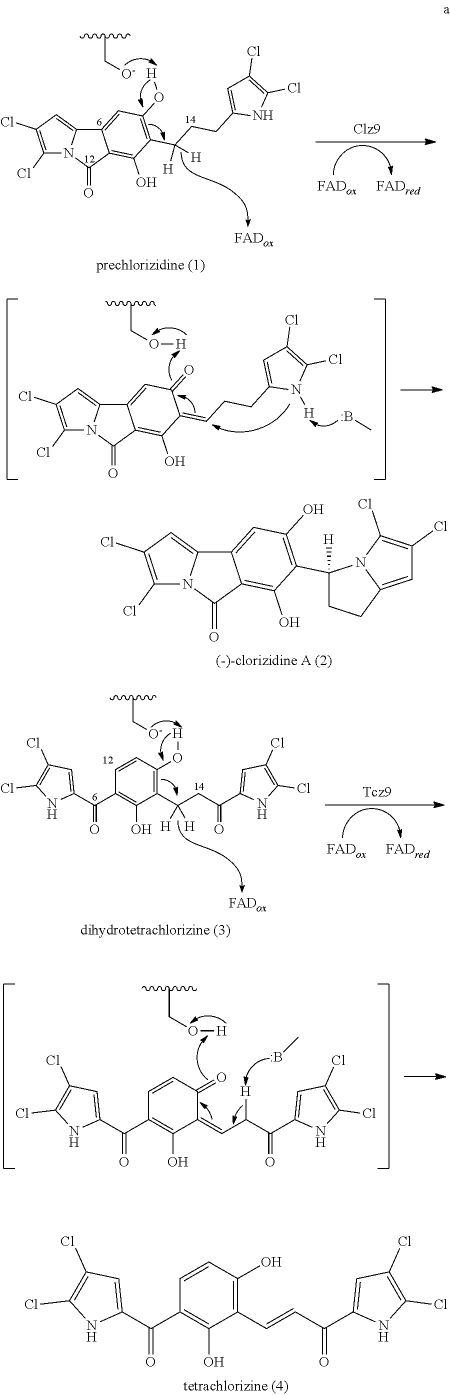

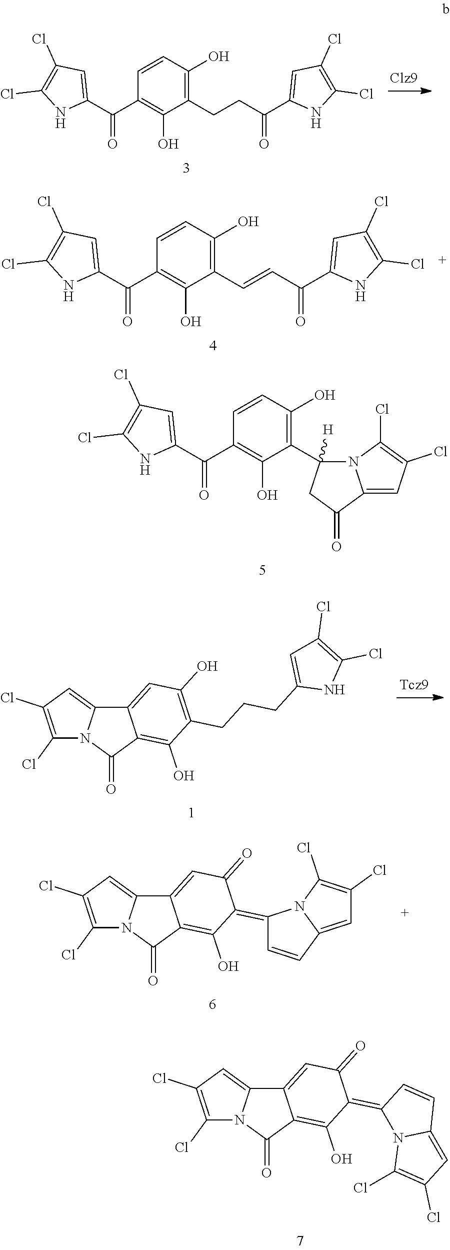 [SMILES: O=C(CCc1c(O)ccc(C(=O)c2cc(Cl)c(Cl)[nH]2)c1O)c1cc(Cl)c(Cl)[nH]1 ; O=C(/C=C/c1c(O)ccc(C(=O)c2cc(Cl)c(Cl)[nH]2)c1O)c1cc(Cl)c(Cl)[nH]1 ; O=C(c1cc(Cl)c(Cl)[nH]1)c1ccc(O)c(C2CC(=O)c3cc(Cl)c(Cl)n32)c1O ; O=C1c2c(cc(O)c(CCCc3cc(Cl)c(Cl)[nH]3)c2O)-c2cc(Cl)c(Cl)n21 ; O=C1C=C2C(=C(O)/C1=c1\ccc3cc(Cl)c(Cl)n13)C(=O)n1c2cc(Cl)c1Cl ; O=C1C=C2C(=C(O)/C1=c1/ccc3cc(Cl)c(Cl)n13)C(=O)n1c2cc(Cl)c1Cl]

BBE-like enzymes are a relatively small family of characterized enzymes with biocatalytic utility.^{42,52,53} The distinctive bicovalent tethering to FAD of BBE-like enzymes has electronic and structural implications for the functions of these enzymes, allowing them to be manipulated to perform a multitude of oxidative reactions with a diverse range of complex substrates found in plants, fungi, and bacteria.⁵³ This plasticity and versatility is enabled by subtle active site variations, as demonstrated by the different activity profiles amongst the plant cannabinoid synthases and two bacterial oxidases, Clz9 and Tcz9. These two groups of BBE-like oxidases are found in completely unrelated organisms and catalyze different reactions with substrates derived from unrelated biosynthetic pathways. Mechanistically, these cannabinoid synthases and bacterial oxidases facilitate hydride abstraction by FAD and phenolic deprotonation within the active site to generate a highly reactive o-QM intermediate. o-QMs typically react with any nucleophile present, but these enzymes direct the o-QM to perform transformative benzylic functionalization reactions chemo-, regio-, and stereoselectively. The delicate balance of promiscuity and selectivity that these enzymes display, coupled with their impressive stability makes them ideal candidates for biocatalytic development.

Clz9 and Tcz9 are promiscuous and versatile, performing several different benzylic functionalization reactions beyond their native function on substrates derived from completely unrelated biosynthetic pathways. The Clz9 and Tcz9 F156/T124 substitution appears to have influence on the difference in reaction selectivity between these two enzymes. Not only can these enzymes catalyze intramolecular nucleophilic addition reactions and dehydrogenations, but they are capable of benzylic hydroxylation and cycloaddition reactions as well. Furthermore, the wild-type enzymes exhibit favorable biocatalytic properties, withstanding temperatures as high as 70° C. and more than 20% DMSO. These enzymes have biomanufacturing potential to produce rare or unnatural cannabinoids in a bacterial heterologous system, a feat yet to be achieved with the plant-derived cannabinoid synthases.

### Definitions

As used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “about”, as used herein, can allow for a degree of variability in a value or range, for example, within 10%, within 5%, or within 1% of a stated value or of a stated limit of a range.

The term “enzyme” or “enzymes”, as used herein, refers to a protein catalyst capable of catalyzing a reaction. Herein, the term does not mean only an isolated enzyme, but also includes a host cell expressing that enzyme. Accordingly, the conversion of A to B by enzyme C should also be construed to encompass the conversion of A to B by a host cell expressing enzyme C.

The term “heterologous” when used in reference to a nucleic acid refers to a nucleic acid that has been manipulated in some way. For example, a heterologous nucleic acid includes a nucleic acid from one species introduced into another species. A heterologous nucleic acid also includes a nucleic acid native to an organism that has been altered in some way (e.g., mutated, added in multiple copies, linked to a non-native promoter or enhancer sequence, etc.). Heterologous nucleic acids can include cDNA forms of a nucleic acid; the cDNA may be expressed in either a sense (to produce mRNA) or anti-sense orientation (to produce an anti-sense RNA transcript that is complementary to the mRNA transcript). For example, heterologous nucleic acids can be distinguished from endogenous plant nucleic acids in that the heterologous nucleic acids are typically joined to nucleic acids comprising regulatory elements such as promoters that are not found naturally associated with the natural gene for the protein encoded by the heterologous gene. Heterologous nucleic acids can also be distinguished from endogenous plant nucleic acids in that the heterologous nucleic acids are in an unnatural chromosomal location or are associated with portions of the chromosome not found in nature (e.g., the heterologous nucleic acids are expressed in tissues where the gene is not normally expressed).

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides. “Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, a “native” nucleic acid or polypeptide means a DNA, RNA, or amino acid sequence or segment thereof that has not been manipulated in vitro, i.e., has not been isolated, purified, amplified and/or modified.

The terms “in operable combination,” “in operable order,” and “operably linked” refer to the linkage of nucleic acid sequences in such a manner that a nucleic acid molecule capable of directing the transcription of a coding region (e.g., gene) and/or the synthesis of a desired protein molecule is produced. The term also refers to the linkage of amino acid sequences in such a manner so that a functional protein is produced.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

As used herein the term “terpene” includes any type of terpene or terpenoid, including for example any monoterpene, diterpene, sesquiterpene, sesterterpene, triterpene, tetraterpene, polyterpene, and any mixture thereof.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The present disclosure can be better understood by reference to the following examples which are offered by way of illustration.

### EXAMPLES

### Example 1: Materials and Methods

### Materials

All solvents purchased from Fisher Scientific were of HPLC grade or higher. Preparative flash column chromatography was carried out on a Teledyne ISCO CombiFlash® Rf+ Lumen™ system using diatomaceous earth for crude extract loading and silica gel 60(EMD, 40-63 μm) for the stationary phase. Preparative HPLC purification was achieved using a Phenomenex Luna C18 column (5 μm, 100×2.0 mm) at a flow rate of 10.0 mL/min, coupled with an Agilent Technologies system composed of a PrepStar pump, a ProStar 410 autosampler, and a ProStar UV detector (Agilent Technologies Inc., CA, USA). NMR spectroscopic data were obtained on a 500 MHz JEOL NMR spectrometer with a 3.0 mm probe. The values of the chemical shifts are described in ppm and coupling constants are reported in Hz. NMR chemical sifts were referenced to the residual solvent peaks (dH 7.26 for CDCb). High resolution LC-MS (HR-LCMS) analysis was conducted on an Agilent 6530 Accurate-Mass Q-TOF MS (MassHunter software, Agilent) equipped with a dual electrospray ionization (ESI) source and an Agilent 1260 LC system (ChemStation software, Agilent) with a diode array detector. Q-TOF MS settings during the LC gradient were as follows: acquisition—mass range acquisition m/z 100-1700, MS scan rate 10/s, MS/MS scan rate 2/s, fixed collision energy 20 eV; source—gas temperature 300° C., gas flow 11 L/min; nebulizer 35 psig, ion polarity negative; scan source parameters—VCap 3000, Fragmentor 100, Skimmer 65, OctopoleRFPeak 750.

Plasmid DNA was isolated from an overnight culture using the QIAprep Spin miniprep Kit (Qiagen, Hilden, Germany) according to the manufacturer's protocol. DNA clean-up after PCR or agarose gel electrophoresis was performed with QIAquick PCR & Gel Cleanup Kit according to the manufacturer's protocol. DNA sequencing was carried out by the Genewiz Sequencing Facility in San Diego, CA.

Sonication of E. coli cells was performed using a 6 mm tip (Qsonica, CT, USA). Protein purification was performed on an AKTApurifier instrument (GE Healthcare, IL, USA) with the modules Box-900, UPC-900, R-900 and Frac-900 with all buffers filtered through a nylon membrane 0.2 μm GDWP (Merck, NJ, USA) prior to use. FPLC data was analyzed with UNICORN 5.31 (Built 743) software. All proteins were purified by Ni²+ affinity chromatography using a 5 mL HisTrap HP (GE Healthcare) column. Proteins were concentrated using Amicon Ultra filters with 50 kDa MWCO (MilliporeSigma). Buffer exchange was performed using an Econo-Pac I0DG desalting column (Bio-rad).

### Site-Directed Mutagenesis Procedures

All Clz9 variants were generated by single primer site-directed mutagenesis of pET-MBP-clz9 plasmid, as disclosed in Mantovani, S. M.; Moore, B. S. Flavin-Linked Oxidase Catalyzes Pyrrolizine Formation of Dichloropyrrole-Containing Polyketide Extender Unit in Chlorizidine A, J Am. Chem. Soc. 2013, 135, 48, 18032-18035, which is incorporated herein by reference.¹ 50 μL PCR reactions were prepared with 1 ng/μL pET-MBP-clz9 plasmid, 0.2 μM primer designated for each mutation (see Table 1), 200 μM dNTPs, 3% (v/v) DMSO, IX Buffer HF, and I U Phusion polymerase. PCR amplification was performed. Following amplification, 1 μL (20 U) Dpnl restriction enzyme (New England Biolabs) was added to the PCR mixture and incubated at 37° C. for 1 h. 2 μL of the digestion mixture was transformed into chemically competent DHI0B E. coli cells.

TABLE 1
(SEQ ID NOs: 10-19)

| Primer Name | Sequence (5′-′3) |
|---|---|
| Clz9 F156T | ggccggcACCtgcccggag |
| Clz9 N400E | cgtcgcgctcGAAtaccacaccgac |
| Clz9 N400L | cgtcgcgctcCTGtaccacaccgac |
| Clz9 T438Y | gcagctacgtcaacTATatcgacctgaccgtcg |
| Tcz9 T124F_ | ccggccggcTTTtgcccgcg |
| F | |
| Tcz9 T124F_ | cgcgggcaAAAgccggccgg |
| R | |
| Tcz9 E368N_ | ctgatggccttcAACtaccgcaccgactg |
| F | |
| Tcz9 E368N_ | cagtcggtgcggtaGTTgaaggccatcag |
| R | |
| Tcz9 T405Y_ | ccgcctacgtcaacTATatcgacctggcc |
| F | |
| Tcz9 T405Y_ | ggccaggtcgatATAgttgacgtaggcgg |
| R | |

All Tcz9 variants were generated with tandem single primer site-directed mutagenesis on pET-MBP-tcz9, as disclosed in Purdy, T. N.; Kim, M. C.; Cullum, R.; Fenical, W.; Moore, B. S. Discovery and Biosynthesis of Tetrachlorizine Reveals Enzymatic Benzylic Dehydrogenation via an ortho-Quinone Methide. J Am. Chem. Soc. 2021, 143, 10, 3682-3686, which is incorporated herein by reference.² Subsequent annealing of amplification products was performed following the procedure outlined by Edelheit et al.³ Briefly, 25 μL PCR reactions were prepared with 70 ng pET-MBP-tcz9 plasmid, 0.2 μM primer (forward or reverse for a designated mutant, see Table 1), 200 μM dNTPs, IX Buffer GC, and I U Phusion polymerase. PCR amplification was performed, and the complementary forward and reverse reactions were combined to a final volume of 50 μL. The combined reactions were denatured and slowly annealed in a thermocycler using the following program: 95° C. for 5 min, 90° C. for 1 min, 80° C. for 1 min, 70° C. for 30 s, 60° C. for 30 s, 50° C. for 30 s, 40° C. for 30 s, hold at 37° C. Following annealing, 1.5 μL (30 U) Dpnl restriction enzyme was added and the mixture was incubated at 37° C. for 2 h. To improve the efficiency of transformation, the Dpnl-digested samples were processed with a PCR clean-up kit (Qiagen), phosphorylated, and ligated prior to transformation. The PCR clean-up consisted of combining the Dpnl-digested sample with 3 V Buffer QG+IV isopropanol, loading onto a micro spin column (Epoch Life Sciences), washing with Buffer PB followed by two washes with Buffer PE, and eluting with 10 μL of warm PCR-grade water. 4 μL of this cleaned and concentrated material was phosphorylated with 0.5 μL T4 polynucleotide kinase (T4 PNK, New England Biolabs) and IX T4 ligase buffer in a final volume of 5 μL by incubating at 37° C. for 30 min and cooling to room temperature. This mixture was brought to a final volume of 10 μL by treating with 0.5 μL T4 DNA ligase (New England Biolabs), 0.5 μL 10× T4 ligase buffer, and 4 μL water and incubated at room temperature for 1 h. The entire 10 μL mix was transformed into 80 μL of chemically competent E. coli DHI OB cells.

In some cases, a Cas9/CRISPR system can be used to create a modification in genomic Tcz9 and/or Clz9 site(s). Clustered regularly interspaced short palindromic repeats (CRISPR)/CRISPR-associated (Cas) systems are useful for, e.g. RNA-programmable genome editing (see e.g., Marraffini & Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is available in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, CA.

An example of an amino acid sequence for a Tcz9 polypeptide
from Actinomycete sp. AJS-327 that can catalyze benzylic
functionalization reactions as described herein is provided at
SEQ ID NO: 1:
MATPSAFSGS VLTPGDDGFE AAQVTWNACY SSRPREVMVC
HDAASVAEAV RSVRERGLPF RVRSGGHSMC GLSNLDDGVI IDLGGLGGVE
LTPDRQTVRI GGGARLADVY NTLWDHRLTV PAGTCPRIGV GGHVLGGGMG
VLSRSRGALV DHLTALEMVD AEGRLLRVSE DENPDLFWAC RGGGGGNFGI
VTAYELRPTP IDDVTIFTVS WTWSQLPDAV RAWQRWLGSA ESRINSFLSL
FPQQQDMVVA FGVFDGPAAD FRPLLAPLTA EVAPEAEVVE EVPFIQAVDT
VEALQGEAAA AEQVRAQGSS AIIANPLNDE ALATLQEFLT DPPSHRAEVA
VYGMGGVIGE RERGDTAFVH RTGLMAFEYR TDWDSPEDDR LNLDWVTRLR
HAMAEHTTGA AYVNTIDLAL ENWLWAYYEE NLPRLMAVKR RYDPENVFHH
PHSIPGSLTA EAARAHGVPE ATLKRLHDDG LLDGPLD
A nucleic acid sequence encoding the Tcz9 polypeptide from
Actinomycete sp. AJS-327 of SEQ ID NO: 1 is provided at
SEQ ID NO: 2:
ATGGCCACCCCATCCGCATTCTCCGGCAGCGTCCTCACCCCCGGT
GACGACGGCTTCGAGGCGGCCCAGGTCACCTGGAACGCCTGCTACTCCTC
CCGCCCCCGGGAGGTGATGGTCTGCCACGACGCCGCGTCCGTCGCCGAGG
CGGTGCGCAGCGTGCGGGAGCGCGGACTGCCCTTCCGGGTGCGCTCCGGC
GGCCACTCGATGTGCGGGCTGAGCAACCTGGACGACGGCGTCATCATCGA
CCTCGGCGGCCTGGGCGGCGTCGAACTCACCCCGGACCGGCAGACGGTGC
GGATCGGCGGCGGCGCCCGCCTCGCCGACGTCTACAACACCCTCTGGGAC
CACCGGCTGACCGTCCCGGCCGGCACCTGCCCGCGGATCGGGGTCGGCGG
CCACGTGCTGGGCGGCGGCATGGGCGTGCTGTCCCGGTCGCGTGGCGCGC
TGGTGGACCATCTGACGGCACTGGAGATGGTGGACGCCGAGGGCCGGCTC
CTGCGCGTCAGCGAGGACGAGAACCCCGACCTGTTCTGGGCCTGCCGGGG
CGGCGGTGGCGGCAACTTCGGCATCGTGACCGCGTACGAGCTGCGGCCCA
CGCCGATCGACGACGTCACCATCTTCACGGTCAGCTGGACCTGGTCGCAG
CTCCCCGACGCGGTGCGGGCCTGGCAGCGGTGGCTGGGCTCGGCGGAGAG
CCGTATCAACAGCTTCCTCTCACTCTTCCCCCAACAGCAGGACATGGTCG
TCGCGTTCGGCGTCTTCGACGGTCCGGCCGCCGACTTCCGGCCGCTGCTG
GCCCCGCTGACCGCCGAGGTCGCCCCGGAGGCCGAGGTCGTGGAGGAGGT
CCCGTTCATCCAGGCCGTGGACACCGTCGAGGCGTTGCAGGGCGAGGCCG
CCGCCGCCGAGCAGGTGCGGGCGCAGGGCAGTTCGGCGATCATCGCGAAC
CCGCTGAACGACGAGGCGCTGGCCACGCTCCAGGAGTTCCTCACCGACCC
GCCCAGCCACCGAGCGGAGGTGGCCGTCTACGGGATGGGCGGCGTCATCG
GCGAACGGGAGCGCGGCGACACGGCGTTCGTGCACCGCACCGGCCTGATG
GCCTTCGAGTACCGCACCGACTGGGACAGCCCCGAGGACGACCGGCTCAA
CCTGGACTGGGTGACCCGGCTGCGGCACGCGATGGCCGAACACACCACCG
GCGCCGCCTACGTCAACACCATCGACCTGGCCCTGGAGAACTGGCTGTGG
GCCTACTACGAGGAGAACCTGCCGCGCCTGATGGCGGTCAAGCGGCGGTA
CGACCCGGAGAACGTCTTCCACCACCCGCACAGCATCCCCGGCTCGCTCA
CCGCCGAGGCGGCCCGGGCGCACGGCGTTCCCGAGGCCACCCTCAAGCGG
CTGCACGACGACGGCCTGCTCGACGGACCGCTGGAC
An amino acid sequence for a Tcz9 polypeptide with a
T405Y mutation of SEQ ID NO: 1 is provided at SEQ ID NO: 3:
MATPSAFSGS VLTPGDDGFE AAQVTWNACY SSRPREVMVC
HDAASVAEAV RSVRERGLPF RVRSGGHSMC GLSNLDDGVI IDLGGLGGVE
LTPDRQTVRI GGGARLADVY NTLWDHRLTV PAGTCPRIGV GGHVLGGGMG
VLSRSRGALV DHLTALEMVD AEGRLLRVSE DENPDLFWAC RGGGGGNFGI
VTAYELRPTP IDDVTIFTVS WTWSQLPDAV RAWQRWLGSA ESRINSFLSL
FPQQQDMVVA FGVFDGPAAD FRPLLAPLTA EVAPEAEVVE EVPFIQAVDT
VEALQGEAAA AEQVRAQGSS AIIANPLNDE ALATLQEFLT DPPSHRAEVA
VYGMGGVIGE RERGDTAFVH RTGLMAFEYR TDWDSPEDDR LNLDWVTRLR
HAMAEHTTGA AYVNYIDLAL ENWLWAYYEE NLPRLMAVKR RYDPENVFHH
PHSIPGSLTA EAARAHGVPE ATLKRLHDDG LLDGPLD
An amino acid sequence for a Tcz9 polypeptide with a T124F
mutation of SEQ ID NO: 1 is provided at SEQ ID NO: 4:
MATPSAFSGS VLTPGDDGFE AAQVTWNACY SSRPREVMVC
HDAASVAEAV RSVRERGLPF RVRSGGHSMC GLSNLDDGVI IDLGGLGGVE
LTPDRQTVRI GGGARLADVY NTLWDHRLTV PAGFCPRIGV GGHVLGGGMG
VLSRSRGALV DHLTALEMVD AEGRLLRVSE DENPDLFWAC RGGGGGNFGI
VTAYELRPTP IDDVTIFTVS WTWSQLPDAV RAWQRWLGSA ESRINSFLSL
FPQQQDMVVA FGVFDGPAAD FRPLLAPLTA EVAPEAEVVE EVPFIQAVDT
VEALQGEAAA AEQVRAQGSS AIIANPLNDE ALATLQEFLT DPPSHRAEVA
VYGMGGVIGE RERGDTAFVH RTGLMAFEYR TDWDSPEDDR LNLDWVTRLR
HAMAEHTTGA AYVNTIDLAL ENWLWAYYEE NLPRLMAVKR RYDPENVFHH
PHSIPGSLTA EAARAHGVPE ATLKRLHDDG LLDGPLD
An amino acid sequence for a Tcz9 polypeptide with a E368N
mutation of SEQ ID NO: 1 is provided at SEQ ID NO: 5:
MATPSAFSGS VLTPGDDGFE AAQVTWNACY SSRPREVMVC
HDAASVAEAV RSVRERGLPF RVRSGGHSMC GLSNLDDGVI IDLGGLGGVE
LTPDRQTVRI GGGARLADVY NTLWDHRLTV PAGTCPRIGV GGHVLGGGMG
VLSRSRGALV DHLTALEMVD AEGRLLRVSE DENPDLFWAC RGGGGGNFGI
VTAYELRPTP IDDVTIFTVS WTWSQLPDAV RAWQRWLGSA ESRINSFLSL
FPQQQDMVVA FGVFDGPAAD FRPLLAPLTA EVAPEAEVVE EVPFIQAVDT
VEALQGEAAA AEQVRAQGSS AIIANPLNDE ALATLQEFLT DPPSHRAEVA
VYGMGGVIGE RERGDTAFVH RTGLMAFNYR TDWDSPEDDR LNLDWVTRLR
HAMAEHTTGA AYVNTIDLAL ENWLWAYYEE NLPRLMAVKR RYDPENVFHH
PHSIPGSLTA EAARAHGVPE ATLKRLHDDG LLDGPLD
An example of an amino acid sequence for a Clz9 polypeptide from
Streptomyces sp. CNH-287 that can catalyze benzylic functionalization
reactions as described herein is provided at SEQ ID NO: 6:
VTADPSSERS DMNEADEVNE VDELSETGQT SGTKGKRPFT
GRVIGPADGE FDEARRVWNE CFAARPKEIV YCADTRDVVR ALREVRQRGG
PFRVRSGGHS MSGLSVLDDG TVLDVSGLDD IQVSEDASTV TVGSGAHLGD
IFRALWARGV TVPAGFCPEI GIAGHVLGGG AGILVRSRGF LSDHLVALEM
VDSEGRIVVA DHDSHHELLW ASRGGGGGNF GIATSFTLRT QPIGDVTLFT
IAWDWDRGAE AIKAWQEWLA TADGRINTLF IAYPQDQDMF AALGCFEGDA
AELEPLIAPL VHAVEPTEKV AETMPWIEAL SFVETMQGEA MKATSVRAKG
NLSFVTEPLG DRAVEEIKKA LAQAPSHRAE VVLYGLGGAV AAKGRRETAF
VHRDAPVALN YHTDWDDEAE DDLNFAWIQN LRASVAAHTE GRGSYVNTID
LTVEHWLWDY YEENLPRLMA VKKRYDPEDV FRHPQSIPVS LTEAEAAELG
IPPHIAEELR AARQLR
A nucleic acid sequence encoding the Clz9 polypeptide from
Streptomyces sp. CNH-287 327 of SEQ ID NO: 6 is provided at
SEQ ID NO: 7:
GTGACGGCGGACCCATCCAGCGAGAGGAGCGACATGAACGAGGCGGACGAGGT
GAACGAGGTGGACGAGTTGAGCGAGACCGGCCAGACGAGCGGCACGAAGGGCAAGCGCC
CGTTCACGGGTCGGGTGATCGGTCCGGCCGACGGTGAGTTCGACGAGGCCCGCAGGGTG
TGGAACGAGTGCTTCGCCGCCCGGCCCAAGGAGATCGTCTACTGCGCGGACACCCGCGA
CGTCGTACGCGCGCTGCGTGAGGTCCGGCAGCGCGGCGGCCCCTTCCGGGTGCGCTCCG
GGGGCCACTCCATGTCCGGGCTGTCCGTACTGGACGACGGCACGGTGCTCGACGTCAGC
GGCCTGGACGACATCCAGGTGAGCGAGGACGCCTCGACTGTGACCGTGGGCAGCGGGGC
GCACCTGGGCGACATCTTCCGGGCGCTGTGGGCCAGGGGCGTCACGGTCCCGGCCGGCT
TCTGCCCGGAGATCGGCATCGCGGGCCATGTGCTGGGCGGCGGCGCGGGAATCCTGGTG
CGCTCGCGCGGATTCCTCAGCGACCACCTGGTGGCGCTCGAGATGGTCGACTCCGAGGG
CCGGATCGTGGTGGCCGACCACGACAGCCACCACGAGCTGCTGTGGGCCTCCCGCGGCG
GCGGCGGCGGGAACTTCGGCATCGCCACCTCCTTCACGCTGCGCACCCAGCCCATCGGC
GACGTCACGCTCTTCACCATCGCCTGGGACTGGGACCGGGGCGCCGAGGCCATCAAGGC
GTGGCAGGAGTGGCTGGCCACCGCCGACGGCCGGATCAACACCCTGTTCATCGCGTACC
CGCAGGACCAGGACATGTTCGCCGCGCTCGGCTGCTTCGAGGGCGACGCGGCCGAACTG
GAGCCGCTCATCGCACCGTTGGTGCACGCCGTCGAGCCGACCGAGAAGGTCGCCGAGAC
CATGCCCTGGATCGAGGCGCTCTCCTTCGTGGAGACCATGCAGGGCGAGGCCATGAAGG
CCACCTCGGTACGGGCGAAGGGCAACCTCTCGTTCGTGACGGAACCGCTGGGCGACAGG
GCCGTCGAGGAGATCAAGAAGGCCCTCGCCCAAGCCCCCAGCCACCGCGCCGAGGTGGT
CCTCTACGGGCTCGGCGGCGCCGTCGCGGCCAAGGGCCGCCGGGAGACCGCGTTCGTCC
ACCGGGACGCCCCCGTCGCGCTCAACTACCACACCGACTGGGACGACGAGGCGGAGGAC
GACCTCAACTTCGCCTGGATTCAGAACCTGCGGGCAAGCGTCGCCGCGCACACCGAAGG
CAGGGGCAGCTACGTCAACACCATCGACCTGACCGTCGAGCACTGGCTCTGGGACTACT
ACGAGGAGAACCTGCCCCGGCTGATGGCGGTCAAGAAGCGGTACGACCCGGAGGACGTC
TTCCGCCACCCGCAGAGCATCCCCGTCTCGCTCACCGAGGCGGAGGCGGCCGAACTGGG
GATTCCGCCGCACATCGCCGAGGAACTCCGGGCCGCCAGGCAGCTGCGG
An amino acid sequence for a Clz9 polypeptide with a F156T
mutation of SEQ ID NO: 6 is provided at SEQ ID NO: 8:
VTADPSSERS DMNEADEVNE VDELSETGQT SGTKGKRPFT
GRVIGPADGE FDEARRVWNE CFAARPKEIV YCADTRDVVR ALREVRQRGG
PFRVRSGGHS MSGLSVLDDG TVLDVSGLDD IQVSEDASTV TVGSGAHLGD
IFRALWARGV TVPAGTCPEI GIAGHVLGGG AGILVRSRGF LSDHLVALEM
VDSEGRIVVA DHDSHHELLW ASRGGGGGNF GIATSFTLRT QPIGDVTLFT
IAWDWDRGAE AIKAWQEWLA TADGRINTLF IAYPQDQDMF AALGCFEGDA
AELEPLIAPL VHAVEPTEKV AETMPWIEAL SFVETMQGEA MKATSVRAKG
NLSFVTEPLG DRAVEEIKKA LAQAPSHRAE VVLYGLGGAV AAKGRRETAF
VHRDAPVALN YHTDWDDEAE DDLNFAWIQN LRASVAAHTE GRGSYVNTID
LTVEHWLWDY YEENLPRLMA VKKRYDPEDV FRHPQSIPVS LTEAEAAELG
IPPHIAEELR AARQLR
An amino acid sequence for a Clz9 polypeptide with a T438Y
mutation of SEQ ID NO: 6 is provided at SEQ ID NO: 9:
VTADPSSERS DMNEADEVNE VDELSETGQT SGTKGKRPFT
GRVIGPADGE FDEARRVWNE CFAARPKEIV YCADTRDVVR ALREVRQRGG
PFRVRSGGHS MSGLSVLDDG TVLDVSGLDD IQVSEDASTV TVGSGAHLGD
IFRALWARGV TVPAGFCPEI GIAGHVLGGG AGILVRSRGF LSDHLVALEM
VDSEGRIVVA DHDSHHELLW ASRGGGGGNF GIATSFTLRT QPIGDVTLFT
IAWDWDRGAE AIKAWQEWLA TADGRINTLF IAYPQDQDMF AALGCFEGDA
AELEPLIAPL VHAVEPTEKV AETMPWIEAL SFVETMQGEA MKATSVRAKG
NLSFVTEPLG DRAVEEIKKA LAQAPSHRAE VVLYGLGGAV AAKGRRETAF
VHRDAPVALN YHTDWDDEAE DDLNFAWIQN LRASVAAHTE GRGSYVNYID
LTVEHWLWD YYEENLPRLM AVKKRYDPED VFRHPQSIPV SLTEAEAAEL
GIPPHIAEEL RAARQLR

### Over-Expression and Purification of Clz9 and Tcz9 Variants

Each Clz9 variant-containing plasmid was transformed into Escherichia coli BL21(DE3). A starter culture was grown overnight in 10 mL of LB media containing 40 μg/mL of kanamycin at 37° C. with overnight agitation. 1 L of TB media containing 40 μg/mL of kanamycin and 100 mg/L of riboflavin was inoculated with the 10 mL starter culture. The cells were grown at 37° C. with shaking (220 rpm) until the culture reached an OD6oo of 0.6. The flasks were then incubated at 4° C. for −30 min without shaking. Then the cultures were induced by adding IPTG to a final concentration of 0.5 mM, the temperature was lowered to 18° C. and the cells were allowed to grow with shaking (200 rpm) for an additional 20 hours. The cells were then harvested by centrifugation at 10,000×g for 10 min at 4° C.

The cell pellet from 1 L of culture was re-suspended in 30 mL binding buffer (50 mM KH2PO4, 150 mM NaCl, 10 mM imidazole, pH 8.0) containing approximately 10 mg of lysozyme. The cells were then lysed by sonication (pulse ‘on’ time 1.0 sec, pulse ‘off’ time 1.0 sec, output level 60%, 30 sec×6 cycles) on ice to make a cell free mixture. The cell debris was removed by centrifugation at 39,000 g for 40 minutes at 4° C. to produce a clarified cell-free mixture. The clarified supernatant was loaded onto a 5 mL Ni-NTA-affinity column pre-equilibrated with binding buffer kept at 4° C. The Ni-NTA-affinity column was then washed with 50 ml wash buffer (50 mM KH2PO4, 150 mM NaCl, 20 mM imidazole, pH 8.0). The protein was eluted from the column with elution buffer (50 mM KH2PO4, 150 mM NaCl, 250 mM imidazole, pH 8.0) at 4° C. The fractions containing protein were pooled and concentrated using an Amicon ultracentrifugal filter (10 kDa MWCO) at 5000×g to a final volume of 2.5 mL. The concentrated sample was buffer exchanged into 100 mM phosphate buffer at pH 7.5 containing 100 mM NaCl and glycerol to a final concentration of 15% using an Econo-Pac 10DG desalting column. Finally, protein aliquots were subjected to flash freezing and stored at −80° C. for future use.

Overexpression and purification protocol for Tcz9 variants were same as that of Clz9. The only difference being Tcz9 variants contain N-terminal MBP tag in addition of His-tag.²

General Procedure for In Vitro Assays with Clz9 and Tcz9 Variants

The in vitro assays were performed following a modified procedure that was previously reported.¹ Assays (0.1 mL) contained 100 mM potassium phosphate buffer pH 7.5, 0.1 mg/mL⁻¹ catalase, 10 μL DMSO, 1 μL of a 20 mM stock solution of substrate and were initiated by addition of 20 μM wild type Clz9 or Tcz9 or their respective variants, unless otherwise altered to investigate the thermal and solvent effects on enzyme activity or product stability during time course experiments. Assays were allowed to incubate for 12 hat 37° C. before quenching with 150 μL acetonitrile. Assays were then centrifuged at 14000×g for 30 minutes and passed through a 0.2 μM filter before injecting 50 μL of the solution for LC-MS analysis at a flow rate of 0.75 mL/min with a mobile phase combination of water+0.1% formic acid (A) and acetonitrile+0.1% formic acid (B) using a gradient as follows: 50% (B), 0 to 2 minutes; 50 to 100% (B), 2 to 10 min; 100% (B), 10 to 17 min; 100% to 50% (B), 17 to 18 min; 50% (B), 18 to 20 minutes.

In Vivo Conversion of CBGA to 15a and 17a with Tcz9_T405Y

A starter culture of Escherichia coli BL21 (DE3) containing the pET-MBP-tcz9_T405Y plasmid was grown overnight in 10 mL of LB media containing 40 μg/mL of kanamycin at 37° C. with overnight agitation. 1 L of TB media containing 40 μg/mL of kanamycin and 100 mg/L of riboflavin was inoculated with this starter culture. The cells were grown at 37° C. with shaking (220 rpm) until the culture reached an OD6oo of 0.6. The flask was then cooled to 18° C. with continuous shaking. Protein expression was induced by adding IPTG to a final concentration of 0.5 mM and 360 mg of CBGA dissolved in 20 mL DMSO was added to the culture (1 M final concentration). The cells were allowed to grow with shaking (200 rpm) for an additional 48 hours. At this time, 500 mL EtOAc was added to the culture and shaken for an additional 2 hours before extracting the organic layer. The aqueous layer was extracted with 500 mL two additional times. The organic layers were pooled, washed with brine, and concentrated under reduced pressure to yield 360 mg of crude extract. The crude mixture was resuspended in 4 mL acetonitrile and filtered prior to preparative HPLC purification at a flow rate of 10 mL/min with a mobile phase combination of water+0.1% formic acid (A) and acetonitrile+0.1% formic acid (B) using a gradient as follows: 80% (B), 0 to 8 minutes; 80 to 100% (B), 8 to 16 min; 100% (B), 16 to 21 min; 100% to 80% (B), 21 to 22 min; 80% (B), 22 to 25 minutes. The solvent was removed to yield 30.1 mg CBCA (compound 15a, 8.3% yield) and 3.7 mg compound 17a (1.0% yield) as clear oils. Spectral data for CBCA matches the reported literature.⁴

15a: ¹H (500 MHz, CDCb) 8 11.71 (br. s, IH); 6.73 (d, IH, J=10.1 Hz); 6.24 (s, IH); 5.48 (d, IH, J=10.1 Hz); 5.09 (t, IH, J=7.1 Hz); 2.88 (dd, IH, J=9.7, 7.7); 2.09 (m, 2H); 1.70 (m, 2H); 1.66 (s, 3H); 1.57 (m, 2H); 1.57 (s, 3H); 1.41 (s, 3H); 1.35 (m, 2H); 1.35 (m, 2H); 0.90 (t, 3H).

17a: ¹H (500 MHz, CDCb) 8 6.67 (d, IH, J=10.1 Hz); 6.32 (s, IH); 5.61 (d, IH, 10.1 Hz); 5.07 (t, IH, J=7.4 Hz); 2.93 (dd, 2H, J=7.8, 11.1 Hz); 2.11 (2H, m); 1.80, (m, 2H); 1.65 (s, 3H); 1.56 (m, 2H); 1.55 (s, 3H); 1.48 (s, 3H); 1.33 (m, 2H); 1.33 (m, 2H); 0.88 (t, 3H).

List of products identified by HPLC analysis from the reactions with 12a, 12b, 12c, and 20 is shown in Table 2.

TABLE 2

| | | | Retention | [M − H]⁻ | UV/Vis |
|---|---|---|---|---|---|
| Name | # | Structure | Time (min) | m/z = | (λₘₐₓ) |
| cannabi- chromenic acid (CBCA)^{a} | 15a | 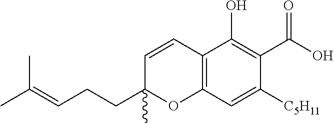 | 11.4 | 357.2058 | 260, 292, 328 |
| TBD | 16a | TBD | 9.5 | 357.2053 | 260, 281, |
| | | | | | 324 |
| TBD^{a} | 17a | TBD | 14.6 | 357.2056 | 274, 294, |
| | | | | | 310, 332 |
| TBD | 18a | TBD | 13.7 | 355.1907 | 260, 329 |
| cannabi- chromevarinic acid (CBCVA) | 15b | 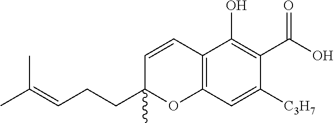 | 10.1 | 329.1745 | 258, 292, 327 |
| TBD | 16b | TBD | 8.0 | 329.1738 | 256, 281, |
| | | | | | 321 |
| TBD | 17b | TBD | 13.3 | 329.1744 | 275, 298, |
| | | | | | 314, 340 |
| TBD | 18b | TBD | 12.2 | 327.1585 | 259, 328 |
| cannabiorci- chromenic acid | 15c | 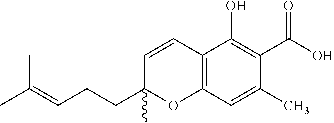 [SMILES: CC(C)=CCCC1(C)C=Cc2c(cc(C)c(C(=O)O)c2O)O1] | 9.0 | 301.1426 | 256, 290, 325 |
| TBD | 16c | TBD | 6.8 | 301.1436 | 258, 278, |
| | | | | | 321 |
| TBD | 17c | TBD | 11.9 | 301.1439 | 275, 300, |
| | | | | | 310, 338 |
| TBD | 18c | TBD | 10.7 | 299.1290 | 259, 326 |
| dauri- chromenic acid (DCA) | 20 | 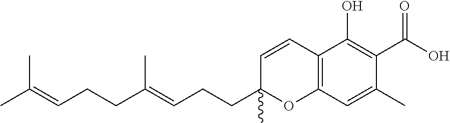 [SMILES: CC(C)=CCC/C(C)=C/CCC1(C)C=Cc2c(cc(C)c(C(=O)O)c2O)O1] | 14.7 | 369.2050 | 258, 290, 325 |

^{a}Purified by preparative HPLC. 1H NMR data acquired.

### Example 2: Site-Directed Mutagenesis of Clz9 and Tcz9

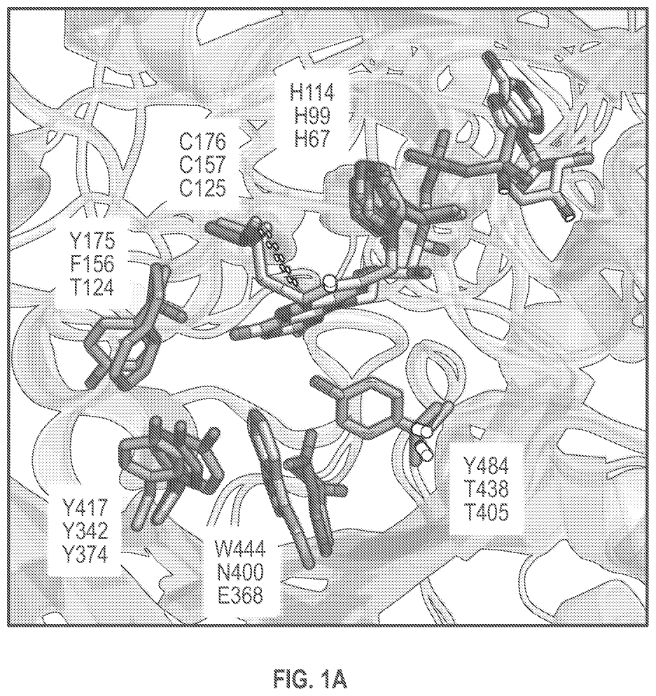

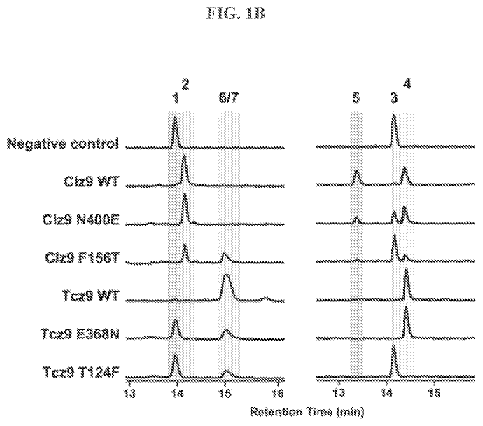

3D protein homology models of Clz9 and Tcz9 were created using AlphaFold and overlaid them with the published crystal structure of FAD-bound tetrahydrocannabinolic acid (THCA) synthase, a plant BBE-like homolog that shares approximately 25% sequence identity with Clz9 and Tcz9.^{18,41} This provided predictions of the relative positioning of FAD in the active site of Clz9 and Tcz9 and evaluate proximal residues that would be critical for catalytic functions. Indeed, both models accurately predict the conserved histidine and cysteine residues necessary for bicovalent attachment (FIG. 1a). Structural analysis of the models reveals three potential residues within the active site of Tcz9 that could be involved in C-14 deprotonation. One of these three residues in Tcz9, Y374, is conserved in Clz9. The two remaining basic residues in Tcz9, T124 and E368, are F156 and N400 in Clz9, respectively, and are hypothesized to play a role in the deprotonation. Thus, we prepared two Tcz9 mutants and two Clz9 mutants to reflect the natural variation observed at these two positions. Clz9 N400E and Tcz9 E368N did not exhibit any noticeable changes compared to the wild type enzymes, other than decreased activity with their non-native substrates (FIG. 1b). Although Clz9 F156T and Tcz9 T124F lowered activity with both substrates, Clz9 F156T did produce a mixture of chlorizidine and the triply oxidized o-QM isomers compound 6 and compound 7. Given this result, the T124 residue in Tcz9 may play a role in differentiating the activity of Clz9 and Tcz9.

### Example 3: Benzylic Hydroxylation Reactions with Non-Native Substrates

Two recent publications examined the utility of o-QMs in biocatalytic applications.³⁶⁻³⁸ Both of these examples employ fungal a-ketoglutarate-dependent (a-KG) non-heme iron oxidases, CitB or ClaD, to perform chemo- and regioselective benzylic hydroxylation reactions. Wild-type CitB and ClaD display impressive promiscuity, hydroxylating more than 20 derivatives of clavatol containing various aromatic substituents. Upon hydroxylation by the a-KGnon-heme iron oxygenases, these products undergo non-enzymatic dehydration to generate an o-QM intermediate, which can further react with additional nucleophiles present in solution, including primary and secondary alcohols, secondary amines, thiols, olefins, and indoles, forming new benzylic C—O, C—N, C—S, and C—C bonds. So far, only methyl groups have been reported to be hydroxylated with these a-KG non-heme iron oxygenases, limiting their utility to enantioselectively functionalize prochiral substrates. We envisioned that BBE-like oxidases Clz9 and Tcz9 could provide an orthogonal approach to functionalize benzylic carbons enantioselectively via direct formation of an o-QM species within the active site of the enzyme.

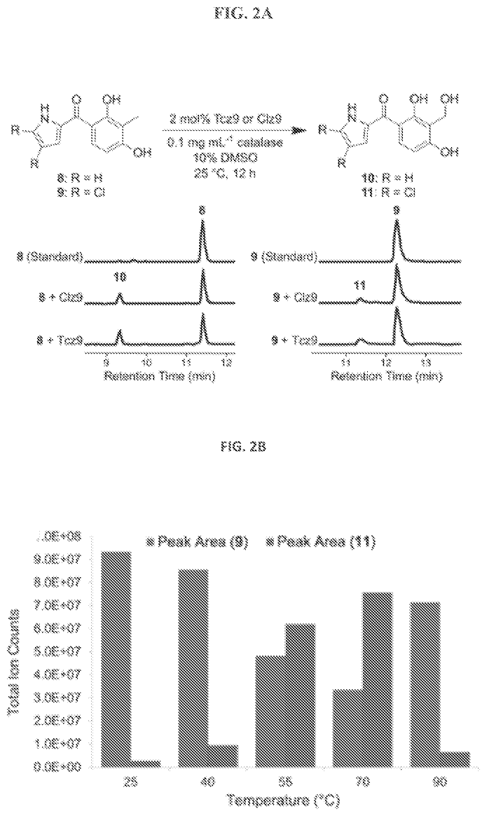

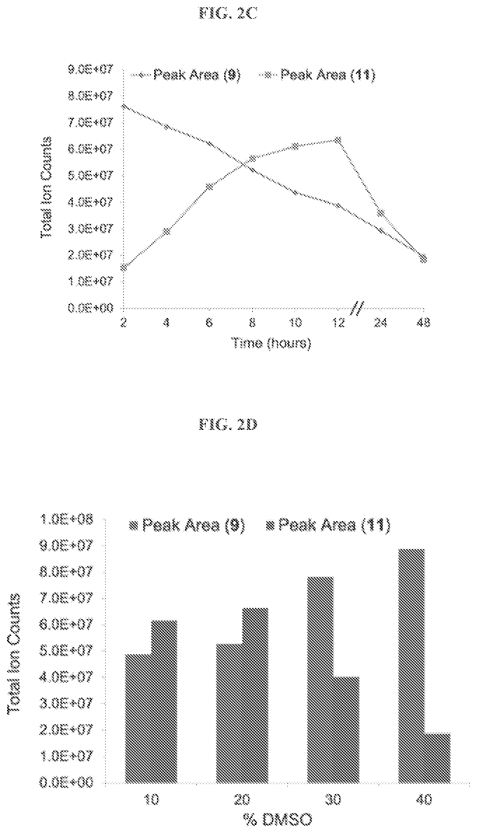

The role of Clz9 and Tcz9 past their cyclization and dehydrogenation functions were developed. Truncated derivatives of dihydrotetrachlorizine (compound 3) were prepared that replaced the pendant dichloropyrrole moiety with a methyl substituent to see if benzylic functionalization would still occur. Surprisingly, both Tcz9 and Clz9 were able to hydroxylate both compound 8 and compound 9 after 12 hours at room temperature, albeit in low quantities (FIG. 2a). Furthermore, the reaction could be performed with the clarified cell free mixture comprising a cell lysate, eliminating the requirement for time-consuming protein purification. Although benzylic hydroxylation is the same reaction outcome as the a-KG non-heme iron oxygenases CitB and ClaD, the mechanism for benzylic hydroxylation with our BBE-like oxidases is fundamentally different. With a-KG non-heme iron oxidases CitB and ClaD, the initial oxygen source is molecular oxygen, which can then undergo non-enzymatic reversible dehydration to reform the o-QM and react with water present in solution;³³ with BBE-like oxidases Clz9 and Tcz9, the oxygen is believed to be sourced directly from water by nucleophilic addition into the enzymatically generated o-QM.

### Example 4: Temperature and Solvent Effects on Tcz9 Activity

Building upon these results, several reaction parameters were examined that might improve the conversion of compounds 9 to 11 with Tcz9. It was observed the in vitro reactions with Clz9 and Tcz9 and their native substrates were slightly faster by performing these reactions in a 37° C. incubator, rather than at room temperature. Surprisingly, wild-type Tcz9 significantly increased the production of compound 11 between 55-70° C., compared to 25° C. (FIG. 2b). Unfortunately, there also appeared to be more substrate consumed above 70° C. without more desired product forming. This is explained by the observation that elevated temperatures promote spontaneous dehydration of the benzylic alcohol product to regenerate the o-QM, which could then degrade or react with other nucleophiles present in solution from the crude cell lysate. Accordingly, the disappearance of substrate at 90° C. with very little product observed could also be explained by possible degradation of the substrate, as no additional significant products could be detected that signified any side reactions were occurring. This pronounced stability at elevated temperatures is unexpected for wild type enzymes from a marine organism, but the bicovalent attachment of the protein to the FAD cofactor might play a role in reinforcing the structural integrity of the active site. This enhanced activity has also been documented with THCA synthase.⁴²

### Example 5: Time Course Analysis Monitoring Reaction Progress of Tcz9 with 9

Guided by these results, the progress of the reaction was monitored every few hours to track substrate consumption and possible product degradation at 55° C. (FIG. 2b). The reaction rate decreases after approximately 8 hours, while substrate concentration continued to decline. After 48 hours, about 66% of the maximum benzyl alcohol product observed had degraded.

Due to the hydrophobic nature of the substrates, we also examined Tcz9's stability in DMSO (FIG. 2d). Initially 10% DMSO was used as a carrier solvent for the substrate, but preliminary data suggests that Tcz9 can withstand upwards of 20% DMSO before activity is significantly affected. Overall, these experiments indicate that optimal reactions can be performed at approximately 55° C. for 8-10 hours in the presence of 10-20% DMSO. The unreacted substrate can be recycled for subsequent in vitro reactions.

### Example 6: In Vitro Cannabinoid Production with Clz9 and Tcz9

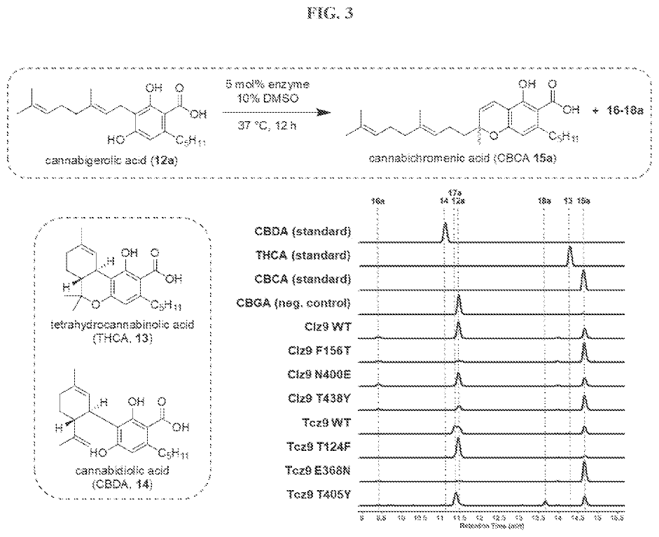

The 2-alkylresorcinol scaffold of dihydrotetrachlorizine (3) and prechlorizidine (1) is shared by other well-known classes of natural products, including phytocannabinoids (commonly known as cannabinoids). In congruence with tetrachlorizine and chlorizidine biosynthesis, the terminal biosynthetic reaction in cannabinoid biosynthesis is also catalyzed by BBE-like enzymes that generate o-QM intermediates. Three enzymes have been characterized from Cannabis sativa L. that catalyze the oxidative cyclization reactions with cannabigerolic acid (CBGA, compound 12a) to produce THCA (compound 13), cannabidiolic acid (CBDA, compound 14), and cannabichromenic acid (CBCA, compound 15a) by their respective THCA, CBDA, and CBCA synthases.^{12, 43, 44} Given the therapeutic and commercial value of cannabinoids, in addition to the uncanny structural, enzymatic, and mechanistic parallels, we were curious if our bacterial BBE-like enzymes associated with marine alkaloid natural products could accept the cannabinoid precursor compound 12a. Indeed, both wild type Clz9 and Tcz9 showed activity with CBGA at 5 mol % after 12 hours at 37° C. (FIG. 3). The major product generated by both Clz9 and Tcz9 at pH 7.5 is compound 15a, but they also produce uncharacterized products compounds 16a and 17a. Interestingly, compound 16a is exclusively produced by the Clz9 variants, while compound 17a is produced exclusively by the Tcz9 variants. High-resolution mass spectrometry confirmed compounds 16a and 17a have the same exact mass as THCA, CBDA, and CBCA ([M-H]″ m/z=357.205; C27H3sQ4), indicating oxidation of compound 12a has taken place. However, the retention times of compounds 16a and 17a differ most significantly from naturally occurring cannabinoids THCA and CBCA, suggesting these products are most likely structurally related to CBDA.

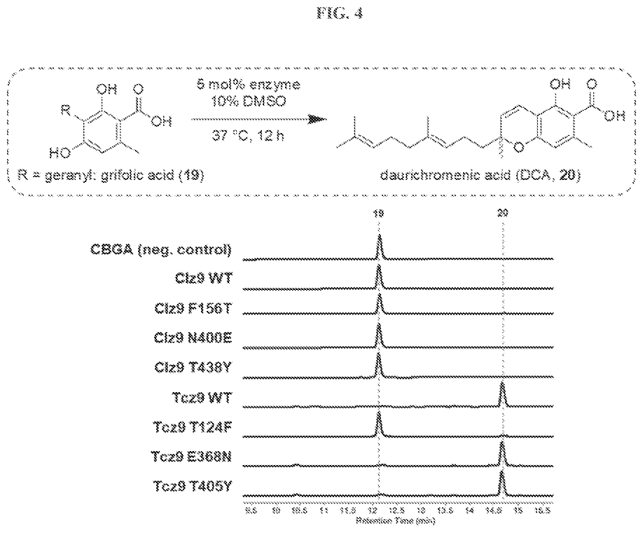

Given these promising results, the four Clz9 and Tcz9 variants were screened and the effect of the F156/T124 and N400/E368 substitutions on catalytic activity with 12a was probed. Clz9 N400E exhibited similar activity to the wild-type enzyme, and the F156T mutant nearly consumed all 12a to produce 15a with minor traces of 16a (FIG. 4a). In contrast, the Tcz9 E368N variant consumed all 12a to produce 15a exclusively, while the T124F variant abolished activity. Two additional variants, Clz9 T438Y and Tcz9 T405Y, were prepared that reflect the THCA synthase Y484 residue that is crucial for catalytic activity in cannabinoid biosynthesis (FIG. 1a).¹⁸ Clz9 T438Y produced compound 15a exclusively, while the Tcz9 T405Y variant produced a 1:1 mixture of 15a and 17a. Tcz9 T405Y also produced a third product 18a with mass 355.1907 ([M-H]″; C27H40Q4) suggestive of a doubly oxidized product; however, this mass does not match any known cannabinoids. Overall, these results suggest that the F156/T124 and N400/E368 residues play a role in substrate binding or positioning within the active site and could be targeted positions for further mutagenesis experiments. Additionally, both Clz9 T438Y and Tcz9 T405Y variants reflecting the tyrosine residue conserved across plant cannabinoid synthases increased conversion of 12a, suggesting that the tyrosine hydroxyl group may be better positioned for phenolic deprotonation of the cannabinoid substrates, compared to the threonine residue in the bacterial BBE-like oxidases.

All isolated cannabinoids from C. sativa L. share a common C10 geranyl chain para to a pentyl or propyl alkyl chain, derived from hexanoyl-CoA or butanoyl-CoA, respectively.⁴⁵ However, cannabinoids with C15 farnesyl chains and methyl substituents have been isolated from other plants and even fungi.^{46,47} There are several reports that the C. sativa cannabinoid synthases accept substrates with various alkyl substituents,^{37,48} but no information has been reported on the effects of altering the prenyl chain length with these enzymes. Biochemical investigations with the recently characterized homolog daurichromenic acid (DCA) synthase from Rhododendron dauricum report this homolog can accept not only geranyl and famesyl prenylated substrates, but also C20 geranylgeranyl prenylated analogs.49 However, DCA synthase is selective for methyl-substituted alkyl groups, exhibiting no activity with 12a. We decided to screen our wild-type Clz9 and Tcz9 enzymes and their variants with three linear cannabinoids with various alkyl and prenyl substituents. Cannabigerovarinic acid (CBGVA, 12b), the propyl analog of compound 12a, exhibited a similar activity profile compared to compound 12a with wildtype Clz9, Tcz9 and all six mutants, producing what we anticipate is cannabichromevarinic acid (CBCVA, compound 15b) as the major product. Similar to the reaction with 12a, the Clz9 enzymes exclusively produce an unknown product 16b, and the Tcz9 enzymes exclusively producing 17b (FIG. 4). Based on the relative retention times, 15b and 16b are most likely the propyl analogs of 15a and 16a.

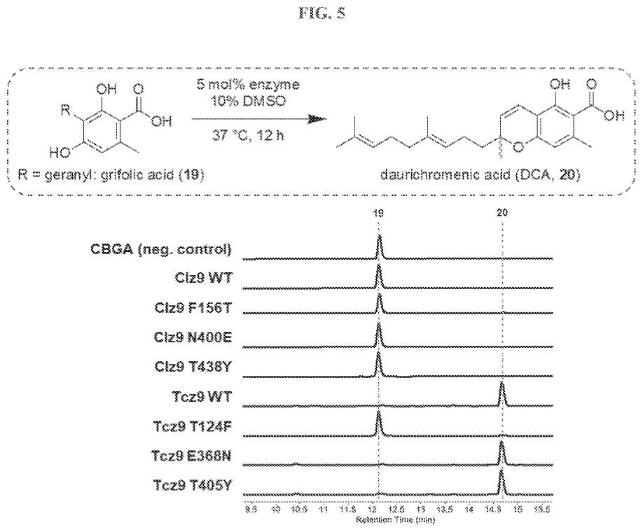

The methyl derivative of compound 12a, cannabigerorcinic acid (CBGOA, compound 12c), exhibited a slightly different activity profile than 12b and 12a (FIG. 4). Tcz9 E368N and T405Y produced compound 15c, which is anticipated to be cannabiorichromenic acid, the methyl analog of compound 15a. Wild-type Tcz9 and Tcz9 T124F produced compound 17c, what is most likely the methyl analog of compounds 17a and 17b (FIG. 4c). Wild type Clz9 and its three variants displayed an activity profile that resembled wild type Tcz9 and its variants, producing a mixture of compounds 15c and 17c with traceable amounts of compound 16c. The final cannabinoid we screened was grifolic acid (compound 19), the only substrate screened at this time with a famesyl chain. Compound 19 exhibited no activity with any of the Clz9 enzymes, but three of the four Tcz9 enzymes fully consumed grifolic acid (compound 19) to produce what is anticipate as DCA (20), given its relative retention time to 19 and the related chromene-containing analogs 15a-c (FIG. 5).

Given the lack of structural information regarding the relationships between THCA synthase, CBDA synthase, and CBCA synthase, it is difficult to predict which residues influence the difference in cyclization and site specificity. However, the fact that Tcz9 T124F completely abolished activity with grifolic acid (19) suggests that the longer famesyl chain may be embedded in the active site and the larger phenyl group may interfere with substrate binding. It is unclear at this time which residues differentiate CBCA synthase from THCA and CBDA synthases, but recent biochemical investigations have determined THCA synthase and CBDA synthase produce higher amounts of THCA (13) and CBDA (14), respectively, at pH values below pH 5.5. At pH values between 5.5 and 8.0, there was greater production of CBCA (15a).⁴²

### Example 7: In Vivo Cannabinoid Production with Tcz9 T405Y

Commercially available cannabinoids are typically sold in their decarboxylated forms, which are more stable and have more therapeutic value. The propyl, methyl, and famesyl derivatives of THCA (13), CBDA (14), and CBCA (15a) are produced in extremely low quantities or sourced from other organisms and are not produced for commercial purposes at this time, and total synthesis routes are prohibitively difficult. The few carboxylated cannabinoids that are commercially available cost several hundred dollars per milligram. Therefore, the most cost-effective approach to verify the identities of these products will be to scale-up reactions to a ten- or hundred-milligram scale and characterize each product by NMR. To streamline the scale-up process, we attempted a whole-cell biotransformation by adding I mM CBGA (12a) to a IL culture of E. coli BL21(DE3) cells overexpressing Tcz9 T405Y. We were able to convert approximately 80% of the CBGA (12a) to the two desired products 15a and 17a over 48 hours with 5× more substrate than we had previously attempted in vitro. To date, THCA synthase has only been successfully heterologously expressed in insect cells or yeast, but not in any bacterial systems.^{18, 37, 50, 51}

The following statements are intended to describe and summarize various features of the invention according to the foregoing description provided in the specification and figures.

### Statements

1. A method of making at least one cannabinoid comprising contacting at least one berberine bridge enzyme like (BBE-like) oxidase with at least one substrate, wherein the substrate comprises a cannabinoid precursor.
2. The method of statement 1, wherein the cannabinoid precursor comprises cannabigerolic acid dihydrotetrachlorizine, prechlorizidine, cannabigerorcinic acid, grifolic acid, or a combination thereof.
3. The method of statement 1, wherein the cannabinoid comprises tetrahydrocannabinolic acid, cannabidiolic acid, cannabichromenic acid, or a combination thereof.
4. The method of statement 1, wherein the at least one berberine bridge enzyme like (BBE-like) oxidase is at least one of:
   a wild type Tcz9 polypeptide comprising an amino acid sequence with at least 95% sequence identity to SEQ ID NO: 1; or
   a mutant Tcz9 polypeptide comprising an amino acid sequence with at least 95% sequence identity to SEQ ID NOs: 3, 4, or 5; or
   a combination thereof.
5. The method of statement 1, wherein the at least one berberine bridge enzyme like (BBE-like) oxidase is at least one of:
   a wild type Clz9 polypeptide comprising an amino acid sequence with at least 95%
   sequence identity to SEQ ID NO: 6; or
   a mutant Clz9 polypeptide comprising an amino acid sequence with at least 95%
   sequence identity to SEQ ID NOs: 8 or 9; or
   a combination thereof.
6. The method of statement 1, wherein the BBE-like oxidase generates o-QM intermediates.
7. The method of statement 1, which is performed in vitro in a cell-free mixture.
8. The method of statement 7, wherein the cell-free mixture is clarified.
9. The method of statement 7, wherein the cell-free mixture is a cell lysate from a cell culture comprising cells expressing the at least one berberine bridge enzyme like (BBE-like) oxidase.
10. The method of statement 1, which is performed within a cell that expresses the at least one berberine bridge enzyme like (BBE-like) oxidase.
11. The method of statement 10, wherein the cell is a host cell comprising an expression system comprising one or more expression cassettes, each expression cassette comprising a promoter operably linked to a nucleic acid segment encoding the at least one BBE-like oxidase.
12. A method of statement 11, wherein the host cell comprises at least one heterologous promotor operably linked to a nucleic acid segment encoding a BBE-like oxidase comprising an amino acid sequence with at least 95% sequence identity to any of SEQ ID NOs: 1, 3, 4, 5, 6, 8, or 9.
13. The method of statement 1, wherein the contacting is performed at about 55° C. to about 70° C.
14. The method of statement 1, wherein the contacting is performed for about 8 to about 10 hours.
15. The method of statement 14, wherein the at least one berberine bridge enzyme like (BBE-like) oxidase and substrate are contacted with about 10% to about 20% DMSO.
16. The method of statement 1, wherein the at least one berberine bridge enzyme like (BBE-like) oxidase is contacted with a solution comprising about 5 mol % cannabigerolic acid substrate at about 37° C.
17. The method of statement 16, wherein the pH of the solution is approximately 7.5.
18. The method of statement 11, wherein the host cell is a bacterium.
19. The method of statement 18, wherein the bacterium is Escherichia coli.
20. A host cell comprising an expression system comprising one or more expression cassettes, each expression cassette comprising a promotor operably linked to a nucleic acid segment encoding at least one berberine bridge enzyme like (BBE-like) oxidase.
21. The host cell of statement 20, wherein the berberine bridge enzyme like (BBE-like) oxidase is from Actinomycete sp. or Streptomyces sp.
22. The host cell of statement 20, the at least one berberine bridge enzyme like (BBE-like) oxidase
   is at least one of:
   a wild type Tcz9 polypeptide comprising an amino acid sequence with at least
   95% sequence identity to SEQ ID NO: 1; or
   a mutant Tcz9 polypeptide comprising an amino acid sequence with at least 95%
   sequence identity to SEQ ID NOs: 3, 4, or 5; or
   a combination thereof.
23. The host cell of statement 20, wherein the at least one berberine bridge enzyme like (BBE-like) oxidase is at least one of:
   a wild type Clz9 polypeptide comprising an amino acid sequence with at least 95% sequence identity to SEQ ID NO: 6; or
   a mutant Clz9 polypeptide comprising an amino acid sequence with at least 95% sequence identity to SEQ ID NOs: 8 or 9; or a combination thereof.

### REFERENCES FOR MATERIALS AND METHODS

(1) Mantovani, S. M.; Moore, B. S. J Am. Chem. Soc. Flavin-Linked Oxidase Catalyzes Pyrrolizine Formation of Dichloropyrrole-Containing Polyketide Extender Unit in Chlorizidine A, 2013, 135, 48, 18032-18035.
(2) Purdy, T. N.; Kim, M. C.; Cullum, R.; Fenical, W.; Moore, B. S. Discovery and Biosynthesis of Tetrachlorizine Reveals Enzymatic Benzylic Dehydrogenation via an ortho-Quinone Methide. J Am. Chem. Soc. 2021, 143, 10, 3682-3686.
(3) Edelheit, O.; Hanukoglu, A; Hanukoglu, I. Simple and efficient site-directed mutagenesis using two single-primer reactions in parallel to generate mutants for protein structure-function studies. BMC Biotechnol. 9: 61 (2009).
(4) Fellermeier, M.; Eisenreich, W.; Bacher, A; Zenk, M. H. Eur. J Biochem. 268: 1596-1604 (2001).

### REFERENCES

1. Nicolaou, K. C. The Emergence of the Structure of the Molecule and the Art of Its Synthesis. Angew. Chemie Int. Ed 52, 131-146 (2013).
2. Madhavan, A, Sindhu, R., Parameswaran, B., Sukumaran, R. K. & Pandey, A Metagenome Analysis: a Powerful Tool for Enzyme Bioprospecting. Appl. Biochem. Biotechnol. 183, 636-651 (2017).
3. Badenhorst, C. P. S. & Bornscheuer, U. T. Getting Momentum: From Biocatalysis to Advanced Synthetic Biology. Trends Biochem. Sci. 43, 180-198 (2018).
4. Devine, P. N. et al. Extending the application of biocatalysis to meet the challenges of drug development. Nat. Rev. Chem. 2, 409-421 (2018).
5. de Rond, T., Asay, J. E. & Moore, B. S. Co-occurrence of enzyme domains guides the discovery of an oxazolone synthetase. Nat. Chem. Biol. 17, 794-799 (2021).
6. Schwizer, F. et al. Artificial Metalloenzymes: Reaction Scope and Optimization Strategies. Chem. Rev. 118, 142-231 (2017).
7. Huffman, M. A et al. Design of an in vitro biocatalytic cascade for the manufacture of islatravir. Science 366, 1255-1259 (2019).
8. Winkler, C. K., Schrittwieser, J. H. & Kroutil, W. Power of Biocatalysis for Organic Synthesis. ACS Cent. Sci. 7, 55-71 (2021).
9. Yang, Y. & Arnold, F. H. Navigating the Unnatural Reaction Space: Directed Evolution of Heme Proteins for Selective Carbene and Nitrene Transfer. Acc. Chem. Res. 54, 1209-1225 (2021).
10. Nazor, J., Liu, J. & Huisman, G. Enzyme evolution for industrial biocatalytic cascades. Curr. Opin. Biotechnol. 69, 182-190 (2021).
11. Rink, E. & Bohm, H. Conversion of reticuline into scoulerine by a cell free preparation from Macleaya microcarpa cell suspension cultures. FEES Lett. 49, 396-399 (1975).
12. Taura, F., Morimoto, S., Shoyama, Y. & Mechoulam, R. First Direct Evidence for the Mechanism of 111-Tetrahydrocannabinolic Acid Biosynthesis. J Am. Chem. Soc. 117, 9766-9767 (1995).
13. Lee, M. H. et al. Structural characterization of glucooligosaccharide oxidase from Acremonium strictum. Appl. Environ. Microbial. 71, 8881-8887 (2005).
14. Noinaj, N. et al. The crystal structure and mechanism ofan unusual oxidoreductase, GilR, involved in gilvocarcin V biosynthesis. J Biol. Chem. 286, 23533-23543 (2011).
15. Kamimura, M. et al. Fungal Ecdysteroid-22-oxidase, a New Tool for Manipulating Ecdysteroid Signaling and Insect Development. J Biol. Chem. 287, 16488-16498 (2012).
16. Nielsen, C. A et al. The important ergot alkaloid intermediate chanoclavine-1 produced in the yeast Saccharomyces cerevisiae by the combined action of EasC and EasE from Aspergillus japonicus. Microb. Cell Fact. 13, 1-11 (2014).
17. Winkler, A, Kutchan, T. M. & Macheroux, P. 6-S-Cysteinylation of Bi-covalently Attached FAD in Berberine Bridge Enzyme Tunes the Redox Potential for Optimal Activity. J Biol. Chem. 282, 24437-24443 (2007).
18. Shoyama, Y. et al. Structure and function of 111-tetrahydrocannabinolic acid (THCA) synthase, the enzyme controlling the psychoactivity of Cannabis sativa. J Mal. Biol. 423, 96-105 (2012).
19. Newhouse, T. & Baran, P. S. If C—H Bonds Could Talk: Selective C—H Bond Oxidation. Angew. Chemie Int. Ed 50, 3362-3374 (2011).
20. Abrams, D. J., Provencher, P. A & Sorensen, E. J. Recent applications of C—H functionalization in complex natural product synthesis. Chem. Soc. Rev. 47, 8925-8967 (2018).
21. Guillemard, L., Kaplaneris, N., Ackermann, L. & Johansson, M. J. Late-stage C—H functionalization offers new opportunities in drug discovery. Nat. Rev. Chem. 5, 522-545 (2021).
22. Liegault, B., Renaud, J.-L. & Bruneau, C. Activation and functionalization of benzylic derivatives by palladium catalysts. Chem. Soc. Rev. 37, 290-299 (2008).
23. Chen, Y., Lie, F. & Li, Z. Enantioselective benzylic hydroxylation with Pseudomonas monteilii TA-5: A simple method for the syntheses of (R)-benzylic alcohols containing reactive functional groups. Adv. Synth. Cata!. 351, 2107-2112 (2009).
24. Genovino, J., Sames, D., Hamann, L. G. & Toure, B. B. Accessing Drug Metabolites via Transition-Metal Catalyzed C—H Oxidation: The Liver as Synthetic Inspiration. Angew. Chemie Int. Ed 55, 14218-14238 (2016).
25. Yazaki, R. & Ohshima, T. Recent strategic advances for the activation of benzylic C—H bonds for the formation of C—C bonds. Tetrahedron Lett. 60, 151225 (2019).
26. Tanwar, L., Borgel, J. & Ritter, T. Synthesis of Benzylic Alcohols by C—H Oxidation. J Am. Chem. Soc. 141, 17983-17988 (2019).
27. Betori, R. C., May, C. M. & Scheidt, K. A Combined Photoredox/Enzymatic C—H Benzylic Hydroxylations. Angew. Chemie 131, 16642-16646 (2019).
28. Bai, W. J. et al. The domestication of ortho-quinone methides. Acc. Chem. Res. 47, 3655-3664 (2014).
29. Van de Water, R. W. & Pettus, T. R. R. o-Quinone methides: Intermediates underdeveloped and underutilized in organic synthesis. Tetrahedron 58, 5367-5405 (2002).
30. Willis, N. J. & Bray, C. D. ortho-Quinone methides in natural product synthesis. Chem. A Eur. J 18, 9160-9173 (2012).
31. Singh, M. S., Nagaraju, A., Anand, N. & Chowdhury, S. ortho-Quinone methide (o-QM): A highly reactive, ephemeral and versatile intermediate in organic synthesis. RSC Adv. 4, 55924-55959 (2014).
32. Ohashi, M. et al. SAM-dependent enzyme-catalysed pericyclic reactions in natural product biosynthesis. Nature 549, 502-506 (2017).
33. Fan, J. et al. Peniphenone and Penilactone Formation in Penicillium crustosum via 1,4-Michael Additions of ortho-Quinone Methide from Hydroxyclavatol to y-Butyrolactones from Crustosic Acid. J Am. Chem. Soc. 141, 4225-4229 (2019).
34. Chen, Q. et al. Enzymatic Intermolecular Hetero-Diels-Alder Reaction in the Biosynthesis of Tropolonic Sesquiterpenes. J Am. Chem. Soc. 141, 14052-14056 (2019).
35. Asai, T. et al. Use of a biosynthetic intermediate to explore the chemical diversity of pseudo-natural fungal polyketides. Nat. Chem. 7, 737-743 (2015).
36. Doyon, T. J. et al. Chemoenzymatic o-Quinone Methide Formation. J Am. Chem. Soc. 141, 20269-20277 (2019).
37. Luo, X. et al. Complete biosynthesis of cannabinoids and their unnatural analogues in yeast. Nature 567, 123-126 (2019).
38. Wu, G. et al. Chemoreactive-Inspired Discovery of Influenza A Virus Dual Inhibitor to Block Hemagglutinin-Mediated Adsorption and Membrane Fusion. J Med Chem. 63, 6940 (2020).
39. Purdy, T. N., Kim, M. C., Cullum, R., Fenical, W. & Moore, B. S. Discovery and Biosynthesis of Tetrachlorizine Reveals Enzymatic Benzylic Dehydrogenation via an ortho-Quinone Methide. J Am. Chem. Soc. 143, 3682-3686 (2021).
40. Winkler, A. et al. A concerted mechanism for berberine bridge enzyme. Nat. Chem. Biol. 4, 739-741 (2008).
41. Jumper, J. et al. Highly accurate protein structure prediction with AlphaFold. Nature 596, 583-589 (2021).
42. Zirpel, B., Kayser, O. & Stehle, F. Elucidation of structure-function relationship of THCA and CBDA synthase from Cannabis sativa L. J Biotechnol.
   284, 17-26 (2018).
43. Taura, F., Morimoto, S. & Shoyama, Y. Purification and Characterization of Cannabidiolic-acid Synthase from Cannabis sativa L. J Biol. Chem. 271, 16411-17416 (1996).
44. Morimoto, S., Komatsu, K., Taura, F. & Shoyama, Y. Purification and characterization of cannabichromenic acid synthase from Cannabis sativa. Phytochemistry 49, 1525-1529 (1998).
45. Gi.ilck, T. & Müller, B. L. Phytocannabinoids: Origins and Biosynthesis. Trends Plant Sci. 25, 985-1004 (2020).
46. Kashiwada, Y. et al. Isolation of rhododaurichromanic acid B and the anti-HIV principles rhododaurichromanic acid A and rhododaurichromenic acid from Rhododendron dauricum. Tetrahedron 57, 1559-1563 (2001).
47. Hashimoto, T., Ngoc Quang, D., Nukada, M. & Asakawa, Y. Isolation, Synthesis and Biological Activity of Grifolic Acid Derivatives from the Inedible Mushroom Albatrellus dispansus. Heterocycles 65, 2431-2439 (2005).
48. Peet, R., Kavarana, M. J., Winnicki, R., Donsky, M. & Sun, M. Chemical Engineering Processes and Apparatus for the Synthesis of Compounds. US 10,472, 1-19 (2019).
49. Iijima, M. et al. Identification and characterization of daurichromenic acid synthase active in anti-HIV biosynthesis. Plant Physiol. 174, 2213-2230 (2017).
50. Carvalho, A., Hansen, E. H., Kayser, 0., Carlsen, S. & Stehle, F. Designing microorganisms for heterologous biosynthesis of cannabinoids. FEMS Yeast Res.
   17, 37 (2017).
51. Thomas, F., Schmidt, C. & Kayser, 0. Bioengineering studies and pathway modeling of the heterologous biosynthesis of tetrahydrocannabinolic acid in yeast. Appl. Microbial. Biotechnol. 104, 9551-9563 (2020).
52. Schrittwieser, J. H. et al. Biocatalytic Organic Synthesis of Optically Pure (S)-Scoulerine and Berbine and Benzylisoquinoline Alkaloids. J Org. Chem. 76, 6703-6714 (2011).
53. Daniel, B. et al. The family of berberine bridge enzyme-like enzymes: A treasure-trove of oxidative reactions. Arch. Biochem. Biophys. 632, 88-103 (2017).

We claim:

1. A method of making at least one cannabinoid comprising contacting at least one berberine bridge enzyme like (BBE-like) oxidase with at least one substrate, wherein the substrate comprises a cannabinoid precursor.

2. The method of claim 1, wherein the cannabinoid precursor comprises cannabigerolic acid dihydrotetrachlorizine, prechlorizidine, cannabigerorcinic acid, grifolic acid, or a combination thereof.

3. The method of claim 1, wherein the cannabinoid comprises tetrahydrocannabinolic acid, cannabidiolic acid, cannabichromenic acid, or a combination thereof.

4. The method of claim 1, wherein the at least one berberine bridge enzyme like (BBE-like) oxidase is at least one of:
a wild type Tcz9 polypeptide comprising an amino acid sequence with at least 95%
sequence identity to SEQ ID NO: 1; or
a mutant Tcz9 polypeptide comprising an amino acid sequence with at least 95%
sequence identity to SEQ ID NOs: 3, 4, or 5; or
a combination thereof.

5. The method of claim 1, wherein the at least one berberine bridge enzyme like (DBE-like) oxidase is at least one of:
a wild type Clz9 polypeptide comprising an amino acid sequence with at least 95%
sequence identity to SEQ ID NO: 6; or
a mutant Clz9 polypeptide comprising an amino acid sequence with at least 95%
sequence identity to SEQ ID NOs: 8 or 9; or
a combination thereof.

6. The method of claim 1, wherein the BBE-like oxidase generates o-QM intermediates.

7. The method of claim 1, which is performed in vitro in a cell-free mixture.

8. The method of claim 7, wherein the cell-free mixture is clarified.

9. The method of claim 7, wherein the cell-free mixture is a cell lysate from a cell culture comprising cells expressing the at least one berberine bridge enzyme like (BBE-like) oxidase.

10. The method of claim 1, which is performed within a cell that expresses the at least one berberine bridge enzyme like (BBE-like) oxidase.

11. The method of claim 10, wherein the cell is a host cell comprising an expression system comprising one or more expression cassettes, each expression cassette comprising a promoter operably linked to a nucleic acid segment encoding the at least one BBE-like oxidase.

12. A method of claim 11, wherein the host cell comprises at least one heterologous promotor operably linked to a nucleic acid segment encoding a BE-like oxidase comprising an amino acid sequence with at least 95% sequence identity to any of SEQ ID NOs: 1, 3, 4, 5, 6, 8, or 9.

13. The method of claim 1, wherein the contacting is performed at about 55° C. to about 70° C.

14. The method of claim 1, wherein the contacting is performed for about 8 to about 10 hours.

15. The method of claim 14, wherein the at least one berberine bridge enzyme like (BBE-like) oxidase and substrate are contacted with about 10% to about 20% DMSO.

16. The method of claim 1, wherein the at least one berberine bridge enzyme like (BBE-like) oxidase is contacted with a solution comprising about 5 mol % cannabigerolic acid substrate at about 37° C.

17. The method of claim 16, wherein the pH of the solution is approximately 7.5.

18. The method of claim 11, wherein the host cell is a bacterium.

19. The method of claim 18, wherein the bacterium is Escherichia coli.

20. A host cell comprising an expression system comprising one or more expression cassettes, each expression cassette comprising a promotor operably linked to a nucleic acid segment encoding at least one berberine bridge enzyme like (BBE-like) oxidase.

21. The host cell of claim 20, wherein the berberine bridge enzyme like (BBE-like) oxidase is from Actinomycete, sp. or Streptomyces sp.

22. The host cell of claim 20, the at least one berberine bridge enzyme like (BBE-like) oxidase is
at least one of:
a wild type Tcz9 polypeptide comprising an amino acid sequence with at least
95% sequence identity to SEQ ID NO: 1; or
a mutant Tcz9 polypeptide comprising an amino acid sequence with at least 95%
sequence identity to SEQ ID NOs: 3, 4, or 5; or
a combination thereof.

23. The host cell of claim 20, wherein the at least one berberine bridge enzyme like (BEE-like)
oxidase is at least one of:
a wild type Clz9 polypeptide comprising an amino acid sequence with at least 95%
sequence identity to SEQ ID NO: 6; or
a mutant Clz9 polypeptide comprising an amino acid sequence with at least 95%
sequence identity to SEQ ID NOs: 8 or 9; or a combination thereof.
